SEQUENCE LISTING (8 sequences)

SEQ ID NO: 1 - DNA, 2126 bp, Zea mays
caaatcccacacacccatgacatcggccggcgatccgatctctatcctgattcctgacacacaagcacggccacggaaccctcgcgcatgcatgctgcctgctgacgcttaccttcgtttcgttttcatggctgctgctgcttattgctgcgagtgcgatgtgcaggcggcagcgggcacagtcctccagtcctcgggtgaggcgatcgtcgccggcgccatgggagggggagtccaccaccaccacccgtgcgtggctgctgatggagatggggccggggccgggcccgggccggccagcgtggaggccgcgttgaggcctcttgtcggcgtcgacgcctgggactactgcgtctactggaggctgtctcctgatcagaggttcttggagatggctgggttctgctgcagcagtcagttcgaggcacagcttccagcgctgggcgacctgcctccatcaatccagctcgactcctcgtctgcagggatgcacgccgaggcaatggtgtccaaccagccgatctggcagagcagccgcgtgccagagctccaaacaggttactccagtggcatggtgcaggagcccgggtccagcggcggcccgaggacgcggctgctggtgcccgtcgccggcggcctcgtcgagctcttcgcggcgagatacatggcggaggaggagcagatggcggagctggtgatggcgcagtgcggggtgccgagcggcggtgaggggggcgcgtggccgccgggattcgcgtgggacggcggcgcctcggacgcgtcgcgtgggatgtacggcgatgcggtgccgccgtcgctcagcctgttcgacgccgccggcagcgtcgcggcggacccgttccaggcggtgcagcaggcgccgggcgccggtggtggtggggtggacgacgtcgccgggtggcagtatgctgctgcggctgggagcgagctggaggcggtgcagctgcagcaggagcagcagccgcgcgatgcggactcggggtccgaggtcagcgacatgcagggggacccagaggacgacggcgacggcgacgcgcaggggcgtggcggcggcaagggcggcgggaagcggcagcagtgcaagaacctcgaggcggagcggaagcggcggaagaagctcaacgagcggctctacaagctcaggtcgctcgtcccgaacatctccaagatggaccgcgcggcgatcctcggggacgccatcgactacatcgtgggcctgcagaaccaggtgaaggcgctgcaggacgagctggaggacccggcggacggcgccggcgcccccgacgtcctcctggaccacccgccgccggcgagcctggtggggctggagaacgacgagtcgccgcccacgagccaccagcacccgctcgccgggaccaagagggcccgtgcggcggcggaggaggaggaggaggagaaggggaatgacatggagccgcaggtggaggtgcggcaggtggaggccaacgagttcttcctgcagatgctgtgcgagcgccggcccgggcgcttcgtccagatcatggactccatcgccgacctgggactggaggtcaccaacgtcaacgtcacctcccacgagagcctcgtcctcaacgtcttccgcgccgccaggcgggacaatgaggtggcagtgcaggcggacagactgagggactcgctgctggaggtgatgcgggagccgtacggcgtatggtcgtcgtcggcgccgcccgtggggatgagcggcagcggcatcgccgacgtgaagcatgacagcgtggacatgaagctcgatggcatcatcgacgggcaggcggcaccgagcgtcgcagtgggggtttcagaggatcactacggcggctacaaccatctcctccaatacctcgcttgatcattatttaattgcgttcgttcatgttgaaagttcgatcaaactatcaaaggatggatcaactaataaaaacgggatccatatataagtaactgtgaattgcgatcattaattgtatgcatacaagcatatggtcgtggattaaagtttgttaattgggttttctcactgcttttctggatctttcttgtgttggttcaaacgagggcggaataataaagctatttcctct

SEQ ID NO: 2 - DNA, 2083 bp, Zea mays
ggctgccgccgcgtggtgcgtttcgcgactgtgtacggtcgttcctggctggatccgcggtgccggcggacggcgcgtggcaggtagcctttggagtcgggaacggggtggtcgtggtgatggaggtggtggaagaggacgtcgctaaaactgggatcgagcggatctactgcgatcactgtaccgtcgccgacttgacagcctgactcctcattaacctgctcggcctgcttctgtgtgttcgtctgaacatgtgtatacattttttcttctacagatgccaatcggctggagtagacaccctgtttgtggaaggaggtaccatttcataattcggaatgaatacaaaacctgcaagcattgtgaccattgtggtcttatggcccagtcgtttggaacacggtgcccgacatgcaaatatgtgatctcctctgatgatccggaagattgggactataggcagttggataatccacgtcacttgctgcatggtattgtacatgacaatgggtttggtcaccttgttcggataaatggcagagagggtggctctagtcttctgacggggattcaactgatgggtttctgggattggctctgcagataccttagagtcagaaaggtctccttgatggatgtctctaagaagtatgaaacagattaccggatcttacatgccatcactactggtcattcatggtatggccaatggggattcaaactcaacaaagggagctttggaattacatcagaagaatacttaaaagctatggacaacctttccttaactccattatcacacttcttcccgcactcccgatatcctcgaaaccagctacaagataccatttcattctaccgatctctttcaaagcaacctctcaccacaattcgtgaactgttcctctatgtgctgggccttgccaccagcaagagttcaaatatgcactatggatcaatgcataaggagcactcacatacccatgtgcaagacacatggcctgacgaggaaataaaacgtgcaacagaaattgctataaaggttcttcgtgctgttgagaaaacaaggtgggtgaccatgcgaatcctaaaggcagccatgtaccattcaattggttcaccgcagctagtggactactgcctcaagacccttggtactagaacaattgatggaatgatggttgcagttcgatgcaacagcgatactaacaccctagagtacaggcttatggatgaacccatcgttctgcccaatgtatccatgccaactcaagaccatcttcgccgtgacataaagttcttgcatgatgctctcctccacccacatacaatgcatccatacaaaccggaaaactgttatgagcatggcaagaggtctgccatggtccttttggactgcaagcaattcacaaagcactatgacctggaacaggagttcttgcctcaaaacccatccatgttgcacctgtggtgtcaagtagaggtgttagaccaggttggcgatccaccttgcataccaccagagctcctaactcttccgcagacagcaactgtgtctgatctgaaggtggaggcaaccagaacattccgtggcatctatctaatgttgcattcctttgtagccgatcggcttgttgactgtggaacggcaagtgagtcaactcaactaaagctcttgtttggggcaaatggaactgttcgcgtccaaggcaggtgtgccagtggtgaacgcagggttgggatttaccggatggagagaggcgtggataaatggacagtgcgttgctcttgtggagccaaggatgatgatggtgagaggatgctgtcttgtgactcttgccatgtgtggcagcacactaggtgtgttgggattagtgatttcgatcaggtgcccaagaaatatgtatgtaactcatgtaaattacttaacaagcgtaagagcagaggtcacggaccagtttataacattggcccaagcaaaagattcaagattggcgcaggtggctttagctctaggtgggggatttttttgaggcctccagctgacatgtaaatatcaataaaatgacagtgagtttgtatg

SEQ ID NO: 3 - DNA, 1410 bp, Zea mays
actaaccatgccgtggctagttaaatgacggggacggggtcacgccttcgttgcgtgcctccacctccccccctcggcgcccccaacgacatgttgttaccgtggctgtggcagccggccggtctccttctccatccatatgtactggcagcatcgtatcacctttttttctgcagcggtgatctcatctaggcgtcggtcagagctctctcgagctcgccagcggtggttggtcgtcgtcgtcgtcgtcgtcgatggggaggccgccgtgctgcgacaaggcgaacgtgaagaaggggccgtggacgccggaggaggacgccaagctgctggcctacacctccacccatggcaccggcaactggaccaacgtgccccaacgagcagggctcaagaggtgcggcaagagctgcaggctgaggtacaccaactacctgcgtcccaacctgaagcacgagaacttcacccaggaggaggaagacctcatcgtcaccctccacgccatgctcggaagcaggtggtctctgatcgcgaaccagctgccgggaaggacggacaacgacgtgaagaactactggaacacgaagctgagcaagaagctgcggcagcgcgggatcgaccccctcacccaccgccccatcgccgacctcatgcacagcatcggcgcgctggccatccgcccgccgcagccggcgacctcccctaacggctccgccgcctaccttcctgcgccggcgctcccgctcgtccacgacgtcgcgtaccacgccgccggaatgctgccgccgacgccggcgccgccccggcaggtcgtcatcgcgcgcgtggaagcggacgcgcccgcgtcgccgacggagcacgggcacgagctcaagtggagcgacttcctcgccgacgacgccgccgccgcggcggcggccgcggccgaggcgcagcagcagctggccgttgttgggcagtaccaccacgaggccaacgccgggagcagcagcgctgcggccggcggtaacgacggttgtggcattgccgtcggcggcgacgacggcgcagcggcgttcatcgacgccatcctggactgcgacaaggagacgggggtggaccagctcatcgccgagctgctggccgacccggcctactacgcgggctcctcctcctcctcctcctcctcgtccgggatgggctgggccggcatgggcctgctgaacgctgattaattaactcaagactgctttagtgtttgctatacgtacttaccatcaattagtatgatggtcaaaccttccaaccggatccattcatatgcttgcacaactctgggagtctgggtgttttcggattacaaattgtacggataattgacgccatttgtgcgtgtgtgtctcattcattttccgaaaaaaaaaaaaaaaa

SEQ ID NO: 4 - DNA, 2004 bp, Zea mays
tccacacgattctgcctgcatattcgtccaaacgactcaagtcaaatgaaaagaacaattttataactaaaattcgagtcaaatgcatttaatcttgaggggttacctaaccctggtgcgcgagggatgtcgattgtgcggattgacatggtaaggtactcttggtcctcatcagcgcccttcttcctgttcgtcggttcgtccgaggttcgtccttgtgggtgcgtgtccaagtgaactcaacttcgtccaacccttctctgtgttttttcgtccgtcatccttgcctggggacaacccctcccttttataggtcgggagaggggtcgcccagcgatggcttccttaggaaggagttgtaaggcaaaggtaaaaccaacgttctacaggggtaaagccacgcgtactcgtgggcccgtagttgcctagatgtctcgtattcacggtggcgaacggcgtggggctacagggcccccaaccgccatcatttaggctatgccgacccatggccttcgcagcctagggctcaaggcggctcgtcgcgttgcgccctgccagtgttgtgcgcactcaggtcgaggggacgcagactataaatgtgtcacagtccgggaggctcgcaggtcatgagtgctatgcgatccaagagttttcgtatgtcatgagtggaaaacggaccaatgctcgcgttgtggctcagactattcatgcggtcggttatttatggcggcttgatctagggtcacgcgtgggatccactcaggtggttttccttcgacatgctcggccctcctatcaggtttcgacgtccgaccctggtctcggtaacgtggtgtttgaccggggacaagctcttttagagttgacgcatccatctcttccagctgaccaacggatctagcgactagggctcttcgttagcgtggtcagagacgtgttctcttaccggcggatcattttccgacaatactaatccaaaggcaggctcatggtggtacatgtccaagtccaatcttctaatgggtatagttaggttatttaaaataataccctaaattctgtcactttcttcattttaatactaatccaagctgccacgacggattgctggtagtggacgagtagtatcggcaaaaaataattactactttttttccggtaaaatttgattactactctacataattagcaaatgaaattaatcacctcttatgcacgttctcactagtaccaagcaacaattcagcttctgcatttcgctaccgttctcttcaatgcgctcgactgatcgcgcacattgcgaagctgtctcttcgtcgtggcctgccattgggattcgagacggggagcaaatgcgcacggcatgcatcgcaatgcaggcaatgaagccgagcagacgcctggccaacctcgatacggcgctgcagcctactacaaatagatgcccaattaacacaacacgcagcgcccgctgtccattcattcaaaacccagccgatcgctctcctccaactaagcagcaagggcagaagcaacgccggcgtgccccacggacgacgctgaattctgcatgtcatcctgcccggccatcgtcaacatgaaggacgacgatggcataggcgctatgggagcggcggtggcgtttgccgccatgggcgtcttcggcatctatttcctgtggcccgtggtgggccccacttcggcggggatgatgatgaaggcgcccggcgccgcagggtgggtcatctgccgcgcggtgttcgaggccaacccgcagttgtattttaccatcctccgcacggccggcgcggcagctgccgctgccacgttcgctgcctgttcgatcgctagctagcgctagctgtgactgtgagcaagtgatcgtcgtaaataaaagatagcgagcgacgagacgagcagcatctgccagtatttccgccgtatgccgatgttgtcggtgttttcccattgaatggagatgttactctatgcgtcgtaattgcc

SEQ ID NO: 5 - protein, 625 aa, Zea mays
MTSAGDPISILIPDTQARPRNPRACMLPADAYLRFVFMAAAAYCCECDVQAAAGTVLQSSGEAIVAGAMGGGVHHHHPCVAADGDGAGAGPGPASVEAALRPLVGVDAWDYCVYWRLSPDQRFLEMAGFCCSSQFEAQLPALGDLPPSIQLDSSSAGMHAEAMVSNQPIWQSSRVPELQTGYSSGMVQEPGSSGGPRTRLLVPVAGGLVELFAARYMAEEEQMAELVMAQCGVPSGGEGGAWPPGFAWDGGASDASRGMYGDAVPPSLSLFDAAGSVAADPFQAVQQAPGAGGGGVDDVAGWQYAAAAGSELEAVQLQQEQQPRDADSGSEVSDMQGDPEDDGDGDAQGRGGGKGGGKRQQCKNLEAERKRRKKLNERLYKLRSLVPNISKMDRAAILGDAIDYIVGLQNQVKALQDELEDPADGAGAPDVLLDHPPPASLVGLENDESPPTSHQHPLAGTKRARAAAEEEEEEKGNDMEPQVEVRQVEANEFFLQMLCERRPGRFVQIMDSIADLGLEVTNVNVTSHESLVLNVFRAARRDNEVAVQADRLRDSLLEVMREPYGVWSSSAPPVGMSGSGIADVKHDSVDMKLDGIIDGQAAPSVAVGVSEDHYGGYNHLLQYLA

SEQ ID NO: 6 - protein, 591 aa, Zea mays
MPIGWSRHPVCGRRYHFIIRNEYKTCKHCDHCGLMAQSFGTRCPTCKYVISSDDPEDWDYRQLDNPRHLLHGIVHDNGFGHLVRINGREGGSSLLTGIQLMGFWDWLCRYLRVRKVSLMDVSKKYETDYRILHAITTGHSWYGQWGFKLNKGSFGITSEEYLKAMDNLSLTPLSHFFPHSRYPRNQLQDTISFYRSLSKQPLTTIRELFLYVLGLATSKSSNMHYGSMHKEHSHTHVQDTWPDEEIKRATEIAIKVLRAVEKTRWVTMRILKAAMYHSIGSPQLVDYCLKTLGTRTIDGMMVAVRCNSDTNTLEYRLMDEPIVLPNVSMPTQDHLRRDIKFLHDALLHPHTMHPYKPENCYEHGKRSAMVLLDCKQFTKHYDLEQEFLPQNPSMLHLWCQVEVLDQVGDPPCIPPELLTLPQTATVSDLKVEATRTFRGIYLMLHSFVADRLVDCGTASESTQLKLLFGANGTVRVQGRCASGERRVGIYRMERGVDKWTVRCSCGAKDDDGERMLSCDSCHVWQHTRCVGISDFDQVPKKYVCNSCKLLNKRKSRGHGPVYNIGPSKRFKIGAGGFSSRWGIFLRPPADM

SEQ ID NO: 7 - protein, 319 aa, Zea mays
MGRPPCCDKANVKKGPWTPEEDAKLLAYTSTHGTGNWTNVPQRAGLKRCGKSCRLRYTNYLRPNLKHENFTQEEEDLIVTLHAMLGSRWSLIANQLPGRTDNDVKNYWNTKLSKKLRQRGIDPLTHRPIADLMHSIGALAIRPPQPATSPNGSAAYLPAPALPLVHDVAYHAAGMLPPTPAPPRQVVIARVEADAPASPTEHGHELKWSDFLADDAAAAAAAAAEAQQQLAVVGQYHHEANAGSSSAAAGGNDGCGIAVGGDDGAAAFIDAILDCDKETGVDQLIAELLADPAYYAGSSSSSSSSSGMGWAGMGLLNAD

SEQ ID NO: 8 - protein, 94 aa, Zea mays
MSSCPAIVNMKDDDGIGAMGAAVAFAAMGVFGIYFLWPVVGPTSAGMMMKAPGAAGWVICRAVFEANPQLYFTILRTAGAAAAAATFAACSIAS

(19) United States
(12) Patent Application Publication
(10) Pub. No.: US 2013/0024985 A1
(43) Pub. Date: Jan. 24, 2013

(54) MALE AND FEMALE STERILITY LINES USED TO MAKE HYBRIDS IN GENETICALLY MODIFIED PLANTS

(71) Applicants: Albert P. Kausch, Stonington, CT (US); Stephen Dellaporta, Branford, CT (US)

(72) Inventors: Albert P. Kausch, Stonington, CT (US); Stephen Dellaporta, Branford, CT (US)

(73) Assignee: Board of Governors for Higher Education, State of Rhode Island and Providence Plantations, Providence, RI (US)

(21) Appl. No.: 13/528,112

(22) Filed: Jun. 20, 2012

Related U.S. Application Data
(63) Continuation of application No. PCT/US2010/062384, filed on Dec. 29, 2010.
(60) Provisional application No. 61/290,592, filed on Dec. 29, 2009.

(51) Int. Cl. A01H 1/02; A01H 5/00; A01H 5/10; A01H 1/04

(52) U.S. Cl. 800/266; 800/260; 800/298; 800/303

(57) ABSTRACT

A method is disclosed for producing a hybrid perennial plant system for plant breeding of co-sexual plants for increased yields and for having increased gene confinement capabilities. The method includes the steps of (a) contacting a first compatible perennial plant with a male vector, wherein the male vector comprises a SL expression cassette to create a plant line (A) with disrupted male development; (b) contacting a second compatible perennial plant with a female vector, wherein the female vector comprises a SL expression cassette to create a plant line (B) with disrupted female development; and (c) crossing plant line (A) with plant line (B) to produce the hybrid perennial plant having increased heterozygocity gene confinement.

### PRIORITY DATA

This application claims priority to U.S. Provisional Patent Application Ser. No. 61/290,592 filed Dec. 29, 2009, the disclosure of which is hereby incorporated by reference in its entirety.

### GOVERNMENT RIGHTS

This invention was made with U.S. Government support under Contract No. DE-FG-36-08GO88070 from the Department of Energy. The Government has certain rights in the invention.

### FIELD OF INVENTION

The present invention generally relates to plant genome modification methods that result in sexually deficient phenotypes that when crossed will produce hybrid sterile plants.

### BACKGROUND OF THE INVENTION

Gene flow between transgenic plants and wild and non-transgenic relatives is widely understood as a major obstacle to genetic improvement of perennial plants. The improvement of many plants by using conventional breeding has historically relied on the identification of a single improved trait within a cultivar and is restricted to germplasm that was capable of sexual crosses to yield fertile offspring. Many important crop plants now can be genetically transformed with genes from other species, even across kingdom barriers. The introduction of cloned genes into plant cells and recovery of stable fertile transgenic plants can be used to make specific modifications in a plant, and has created the potential for genetic engineering of plants for crop improvement. Genetic modifications by plant transformation allow stable alterations in biochemical processes that direct traits such as increased yield, disease and pest resistance, increased vegetative biomass, herbicide tolerance, nutritional quality, drought and stress tolerance, as well horticultural qualities such as pigmentation and growth, and other agronomic characteristics for crop improvement. In these methods, foreign DNA was introduced into the eukaryotic plant cell, followed by isolation of cells containing the foreign DNA integrated into the cell's DNA, to produce stably transformed plant cells. The problem is that gene flow can occur between genetically modified crops and their wild relatives. Therefore, synthetic lethality of male and female reproduction serves as an important tool for gene confinement and breeding.

One method to create synthetic lethality (SL) would be to utilize techniques to create stable knock out mutations in fertility genes. While there are many method to disrupt fertility, including RNAi and RNAases, one of the more promising approaches included artificial DNA restriction enzymes, called zinc-finger nucleases (ZFNs) that were created by fusing a DNA-binding domain, encoding zinc finger proteins, to a DNA-cleavage domain, such as FOK 1, that is capable of generating double stranded breaks and subsequent repair by non-homologous end joining (NHEJ). ZFN domains can be tailor-made to target any desired DNA sequence(s) to cause stable deletions in their targets and this characteristic allows SLs to target any unique sequence within a complex genome. Recently, the use of ZNF technology has been demonstrated to be useful for the creation of specific deletions and insertions in plants. By taking advantage of endogenous DNA repair machinery, these reagents can be used to precisely alter the genomes of higher organisms. Additional methods to create synthetic fertility lethality are described.

The problem of gene flow is particularly important to the genetic improvement of perennial plants, such as those currently developed by the DOE for biofuels, such as switchgrass, poplar, willow and others. While improved genetic traits can be modified in these plants, their ability to outcross with wild and native populations creates environmental risk in some cases, and costly regulatory concerns. The utilization of energy crops produced on American farms as a source of renewable fuels is a concept with great relevance to current ecological and economic issues on both national and global scales. Development of a significant national capacity to utilize perennial forage crops, such as switchgrass (Panicum virgatum, L., Poaceae), as biofuels could provide independence from foreign oil, a cleaner source of energy for road fuel to diminish greenhouse gas emissions, benefit the agricultural economy by providing an important new source of income for farmers, and allow for more productive use of land currently within the Conservation Reserve Program (CRP). In addition energy production from perennial cropping systems, which are compatible with conventional farming practices, would help reduce degradation of agricultural soils, lower national dependence on foreign oil supplies, and reduce emissions of greenhouse gases and toxic pollutants to the atmosphere.

One drawback that arises regarding transgenic improvement of perennials, such as switchgrass and other plant based biofuels, is the biological fact that when plants produce flowers, gene flow can occur to wild and non-transgenic plants. Limitations in the current availability of renewable resources influence the need for the development of dedicated feedstock crops as a source of bioenergy from biomass. There are global economic, political, and environmental pressures to increase biofuel production and utilization, to offset gasoline and diesel fuel use, especially in the transportation sector. Many governments, including the US government, have issued increasingly aggressive targets for renewable energy over time; these will certainly require new dedicated feedstocks and fuel platforms. Current strategies for liquid fuel production are focused on using ethanol as a gasoline additive and offset, which utilize fermentation of plant-produced starches and sugars mostly from maize grain and sugar cane to ethanol. It is doubtful whether sufficient amounts of these feedstocks can be supplied without impacting the agricultural sector and harming the environment. Thus, it is necessary to develop biofuels produced from dedicated non-food cellulosic feedstocks such as switchgrass, Energy Cane, sorghum, Miscanthus, willow, and poplar or develop enzymes required for biomass degradation to release fermentable sugars in non-food/non-feed crops like tobacco that yield as high as 44 metric tons of biomass per acre annually.

Genetic improvement of food and fiber crops has been accelerated through biotechnology and breeding. This same model should be useful for improving bioenergy feedstocks. Rapid genetic improvement of the most promising perennial grass feedstocks, such as switchgrass and Miscanthus, which are not highly domesticated, are thus anticipated by molecular assisted breeding methods. In addition, biofuel-specific traits such as production of glycosyl hydrolases, biopolymers, cell wall biosynthesis proteins for increased cellulose, and decreased lignin can be engineered to increase fuel production per acre. The use of biotechnology to improve any feedstock is in its infancy, yet it offers significant potential to improve the utility and production of these cropping systems. In addition, there are also rapidly growing genomics resources for feedstocks. The draft genomes of hybrid poplar and sorghum have been published. The Joint Genome Institute (JGI) of the U.S. Department of Energy (Walnut Creek, Calif.) has performed shotgun sequencing of the switchgrass genome. There are also several metagenome projects designed to discover new enzymes from cell wall degrading bacteria and fungi. However, in contrast to the situation for food and fiber crops, it might not be economically feasible to deploy cellulosic feedstocks without addressing both the need to improve the agronomic aspects of their growth on a commercial scale, as well as the recalcitrance problem (i.e., the integrity of the cell walls that makes digestion to simple sugars difficult and costly) in the feedstock itself. Transgenic input traits for traditional row crops have had tremendous economic and environmental benefits, but maize, soybean, cotton and canola were already successfully cultivated in a mature industry prior to biotechnological innovations. In contrast, cellulosic feedstocks have yet to be widely grown, and all suffer from the recalcitrance problem. Currently, the cost of pretreatment and exogenous enzymatic digestion to break down cell walls renders cellulosic biofuels uncompetitive with starch-based ethanol. Likely, some combination of transgenes will be needed to address the recalcitrance problem and also to increase current yields and establish sustainability. Because of this need for a biotechnological approach to both establish feedstock agriculture and to solve processing problems, perhaps the greatest hurdle standing in the way of the commercialization of transgenic feedstocks and their wide scale deployment involves environmental regulation and biosafety.

Even though there is an absence of documented risks among commercially-grown transgenic crops, commercial-scale production of certain combinations of transgenic traits and crops could potentially lead to undesirable environmental and agricultural consequences. This is because many of the traits that are beneficial to the feedstock industry potentially impact plant fitness and the ability of the plants to compete for resources. Thus in all probability the main biosafety and regulatory issue that will receive immediate scrutiny among transgenic bioenergy feedstocks will revolve around transgene flow from cultivated fields to non-transgenic sexually compatible conspecifics and congeners. Thus, to realize the full potential of agricultural biotechnology for dedicated energy crops enhancement, the ecological, economic, as well as commercial impacts of gene flow must be addressed.

As an open pollinated obligate outcrossing species, switchgrass expresses tremendous genetic diversity, with wide variations in its basic chromosome number (2n=18), typically ranging from tetraploid to octoploid. Morphologically switchgrass in its southern range can grow to more than 3 m in height, but what is most distinctive is the deep, vigorous root system, which may extend to depths of more than 3.5 m. It reproduces both by seeds and vegetatively and, with its perennial life form, a stand can last indefinitely once established. Standing biomass in root systems may exceed that found aboveground, giving perennial grasses such as switchgrass, an advantage in water and nutrient acquisition even under stressful growing conditions.

Physiologically, switchgrass, like maize, is a C4 species, fixing carbon by multiple metabolic pathways with high water use efficiency. In general C4 plants such as grasses will produce 30% more biomass per unit of water than C3 species such as trees and broadleaved crops and grasses and are well adapted to the more arid production areas of the mid-western US where growth is more limited by moisture supply.

To date switchgrass has been bred primarily to enhance its nutritional value as a forage crop for livestock. Thus, it has been managed primarily as a hay crop for which high leaf to stem ratio and high nutrient content are important. These targets are quite different from the criteria for biofuels crops for which high biomass yield, high cellulose, and low ash content are important for high energy conversion and low contamination of combustion systems.

### Regulatory Issues for Perennial Transgenic Plants

Currently, the USDA-APHIS-BRS regulates the environmental release of transgenic plants on a case-by-case basis. Permits are required for all non-deregulated transgenic plants to be grown outside of containment greenhouses. The value of BRS' to both biosafety and innovation in transgenic field testing is apparent in as much that transgenic releases in the US do not require costly permitting or undue paperwork. However, permits often are accompanied by additional requirements. For example, in the field testing of transgenic switchgrass, are required to prevent flowering and set seed; i.e., by the mechanical removal of flowers prior to anthesis. BRS considers the planting of transgenic switchgrass, a plant with which they have little experience, to be a case that required the imposition of a stringent set of precautions to avoid gene flow when the first field tests were performed; even though the transgenic contain only non-herbicide selectable and scorable marker genes.

The process of US deregulation includes lengthy reviews and data collection spanning different environments over several years with consideration of several factors including biology, geography and ecology of the plant, the genes and traits of interest, the possibility of gene flow to wild and non-transgenic relatives, the possibility of weediness or invasiveness, and unintended consequences to other organisms. It is important to assess individual bioeneregy feedstock species independently and to evaluate the introduced traits or characteristics to determine if they could enhance the vigor or invasiveness of wild or weedy relatives or have other detrimental effects. While some traits may pose relatively few risks (e.g., herbicide tolerance), others might have the potential for unintended consequences and invasiveness (e.g., drought and pest tolerance). Most of the next-generation dedicated energy crops will be perennial trees and grasses. Many species that are being seriously considered to play a major role in the developing biofuels industry have wild relatives in the regions where they will be grown. In addition, for some prominent feedstocks, such as switchgrass, there is an absence of data on gene flow. The regulatory data requirements or constraints for gene flow are still unclear. While one may assume that transgene containment is the goal, acceptable levels of transgene escape need to be practically defined. Considering the cost of deregulation and the subsequently imposed market restrictions, the risks and benefits of some regulatory requirements may need to be reconsidered i.e., modified without unduly compromising safety. For example, deregulation of the transgenic process itself, creation of regulatory classes in proportion to potential risk, exemption of selected transgenes and classes of transgenic modifications, and elimination of event-specific basis of transgenic regulation.

### SUMMARY OF THE INVENTION

While there are many strategies currently in development for gene confinement, as described above, all suffer from serious set backs. None of these methods has been field tested or reduced to commercial practice and all suffer from the criticism of incomplete and or unstable confinement. The current invention describes a method to overcome these drawbacks by inducing stable mutations in floral development in male and female lines to create sterile hybrids which overcome all obstacles exemplified in the prior art. In addition, this approach facilitates the breeding process.

The currently available strategies for transgene biocontainment that are currently under development have been described above. There are limitations to most of these strategies, for example, most notably and obviously, those associated with physical, spatial, mechanical and temporal containment. In addition, some of the more sophisticated biotechnology methods are not perfected or adapted for bioenergy feedstocks and have not been field tested or introduced into commercial development. Biotechnology specifically for biocontainment is in the early stages of development and there are many choices with regard to components. Pollen-sterility has been accomplished in a number of species but there are not many systems that have proven to be effective. Certainly, additional male-sterility systems are needed. Male sterility should be sufficient for mitigating gene flow in many cases, as wild type crosses would produce progeny that would also be male sterile, but transgenes can be silenced or somaclonally affected. Very little is known about the frequency of reversion of these mechanisms (i.e. ribonucleases, barnase, etc.) to fertile phenotypes. CMS systems would provide a similar level of biocontainment, but again, additional technologies are needed to enable the necessary freedom-to-operate that would spur development. Any system currently suggested has not been rigorously tested in the field for the species of interest.

The GeneSafe technology and other seed-based GURTS offer conditional lethality which can be chemically induced to prevent flowering or seed development. Currently these approaches are considered to be the best and only strategies that could be deployed to prevent seed based gene flow. However, these technologies require complete biological induction and have human management drawbacks. It also might be required to include failsafe and backup mechanisms to prevent reversion.

Methods have been developed for generating male and female sterile lines using three methods using synthetic lethality including 1) male and female specific cell ablation that results in sterile hybrids, 2) synthetic lethality directed cell ablation for reproductive specific male and female genes that result in male and female sterile lines that can be used for breeding; and, 3) creation of stable knockout mutations in genes required for fertility whereby using these lines from either method or in concert in crosses will create hybrid progeny that will be completely sterile. In addition, these approaches will create populations significant to breeding efforts in these crops and other plants. The implementation of controllable total sterility in genetically modified transgenic perennial plants will (1) control gene flow in transgenic plants eliminating or diminishing potential risks of transgene flow, (2) provide a robust breeding strategy for these types of plants and many others, and (3) allow the necessary gene stacking requirements for further genetic modification. While the examples here focus on switchgrass for applications in biofuels feedstock development, a similar strategy can also be applied to other plant species when developing genetically engineered products using recombinant DNA technology. The synthetically lethality may include including ZFN's.

An object of the invention is to provide a unique and non-obvious approach to gene containment by using male and female sterile lines to create sterile hybrid plants to control gene flow in genetically modified plans and facilitate breeding.

Another object of the invention is to devise a method of producing a hybrid perennial plant having increased gene confinement.

The efficient, industrial-scale production of hybrid maize is made possible because of its unisexual flowers, one of our nation's most important agricultural traits. In maize, unisexual traits are controlled by a sex determination (SD) pathway, a set of genetic instructions that results in exclusively staminate or pistillate florets. Unlike other complex developmental pathways, such as the homeotic control of flower development, SD is controlled by a relatively small set of genes that act late in floral development to eliminate or arrest the maturation of preformed floral organ initials. As a complete genetic and molecular definition of the major genes controlling maize SD pathway, extending unisexual traits to related cosexual cereals is possible. The current invention uses the information of maize SD genes in cosexual grasses to disrupt these pathways and extend unisexuality pathways to cosexual crop species such as rice, wheat, oats, sorghum and switchgrass. This same approach can be applied to other cosexual perennial plants, including trees. The extension of sex determination systems to other crops will provide an immediate impact on yield and for the first time permit large-scale hybrid seed production to enhance plant vigor and yield.

Custom DNA restriction enzymes, called zinc-finger nucleases (SLs examples), are created by fusing a sequence-specific zinc finger DNA-binding domain to a DNA-cleavage domain, such as Fok I. Fok I contains two functional domains, one that binds DNA and another that cleaves to create double stranded breaks in DNA adjacent to the DNA binding site. SL domains fused to the nuclease domain of Fok I can be engineered to recognize specific target sequence(s) and to create a sequence-specific double stranded cleavage. Repair of double stranded breaks in plants and animals is primarily mediated through non-homologous end joining, NHEJ, creating a stable deletion at the target site. This characteristic allows SL to target any unique sequence within a complex genome. Artificial SLs have now been successfully used in a number of plant species such as tobacco Arabidopsis, and maize.

This invention applies the basic science of SD-related genes to develop hybrid technologies. Thus, another object of this invention is to use existing genomic resources and SL technology, to: 1) create loss-of-function mutations in SD orthologs of switchgrass and related species; 2) to identify and map unisexual traits in related species; and 3) to specifically alter the pathways of stamen and pistil maturation to create unisexual traits in switchgrass. The implementation of unisexuality in cosexual grasses will provide a robust breeding platform to stimulate the development of hybrid seed industry in crops important to biofuels development. This strategy demonstrates a “proof-of-principle” initially in switchgrass, a major developing biofuels crop. Yet, the technologies and strategies developed for switchgrass should have broad application to all cosexual grasses including crops important to the agriculture. In addition, the SL vectors and reagents developed in this patent will have utility for additional applications in cereals such as targeted transgene integration and gene stacking in rice and other cereals such as sorghum, sugarcane, millets and other species important to the agriculture.

These and other objects, features and advantages of the present invention will become apparent in light of the following detailed description of preferred embodiments thereof, as illustrated in the accompanying drawings.

### BRIEF DESCRIPTION OF THE DRAWINGS

The following description may be further understood with reference to the accompanying drawings in which:

FIG. 1 shows a breeding strategy utilizing male sterility to recover rare hybrids in switchgrass;

FIGS. 2A and 2B show test constructs for PHG 018 and SL knockouts;

FIG. 3 shows hybrid strategies for sterility constructs;

FIG. 4 shows PCR test results from DNA samples;

FIG. 5 show a Southern Blot of the DNA samples of transgenic switchgrass; and

FIG. 6 shows gene constructs for total sterility for both male and female plant lines.

### DETAILED DESCRIPTION OF THE DISCLOSED EMBODIMENTS

Described herein are the use of these reagents, and others to specifically target genes required for floral development to create mutations which ablate fertility. The ability to control sexual development in plants is a major advantage to plant breeding and the control of gene flow. The technology to control the development of floral reproductive structures allows for the creation of sterile lines, and provides a method for the prevention of gene flow. Genes can be introduced into plants that confer desirable traits such as, drought and stress tolerance, insect and pest resistance, as well as traits for enhancing biofuel production, such as increased vegetative biomass and prolonged vegetative growth. One problem is that the development of fertile reproductive structures results in a risk of undesirable gene flow to non-transgenic and wild plants. Disclosed herein are methods for generating and using male and female sterile lines as a breeding tool and for the purpose of controlling gene flow from transgenic plants.

### ABBREVIATIONS AND TERMS

The following explanations of terms and methods are provided to better describe the present disclosure and to guide those of ordinary skill in the art in the practice of the present disclosure. As used herein, the singular forms “a” or “an” or “the” include plural references unless the context clearly dictates otherwise. For example, reference to “a transgenic plant” includes one or a plurality of such plants, and reference to “the floral-specific promoter” includes reference to one or more floral-specific promoters or their homologues and equivalents thereof known to those skilled in the art.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

Anther-specific gene: A gene sequence that is primarily expressed in the anther, relative to expression in other plant tissues. Includes any anther-specific gene whose malfunction or functional deletion results in male-sterility. Examples include, but are not limited to: anther-specific gene from tobacco (GenBank Accession Nos. AF376772-AF376774), and Osg4B and Osg6B (GenBank Accession Nos. D21159 and 21160).

Anther-specific promoter: A DNA sequence that directs a higher level of transcription of an associated gene in anther tissue relative to the other tissues of the plant. Examples include, but are not limited to: anther-specific gene promoter from tobacco (GenBank Accession Nos. AF376772-AF376774), and the promoters of Osg4B and Osg6B (GenBank Accession Nos. D21159 and D21160).

Asexual: A plant lacking floral structures such that it is incapable of participating either as a male or female parent in sexual reproduction and propagates vegetatively.

Comprises: A term that means “including.” For example, “comprising A or B” means including A or B, or both A and B, unless clearly indicated otherwise.

Deletion: The removal of a sequence of a nucleic acid, for example DNA, the regions on either side being joined together.

Desirable trait: A characteristic which is beneficial to a plant, such as a commercially desirable, agronomically important trait. Examples include, but are not limited to: resistance to insects and other pests and disease-causing agents (such as viral, bacterial, fungal, and nematode agents); tolerance or resistance to herbicides; enhanced stability; increased yield or shelf-life; environmental tolerances (such as tolerance to drought, heat, chilling, freezing, excessive moisture, salt stress, or oxidative stress); male sterility; and nutritional enhancements (such as starch quantity and quality; oil quantity and quality; protein quality and quantity; amino acid composition; and the like). In one example, a desirable trait is selected for through conventional breeding. In another example, a desirable trait is obtained by transfecting the plant with a transgene(s) encoding one or more genes that confer the desirable trait to the plant.

Floral deficient: A plant that is lacking, or is functionally deficient in, one or several parts of the male or female structures contained within a single flower or inflorescence effectively resulting in either male or female sterility.

Floral-specific gene: A gene sequence that is primarily expressed in floral tissue or during the transition from a vegetative to floral meristem, such as the tapetum, anther, ovule, style, or stigma, relative to the other tissues of the plant. Includes any floral-specific gene whose malfunction or functional deletion results in sterility of the plant either directly or by preventing fertilization of gametes through floral deficiencies.

Floral-specific promoter: A DNA sequence that directs a higher level of transcription of an associated gene in floral tissues or during the transition from vegetative to floral meristem relative to the other tissues of the plant. Examples include, but are not limited to: meristem transition-specific promoters, floral meristem-specific promoters, anther-specific promoters, pollen-specific promoters, tapetum-specific promoters, ovule-specific promoters, megasporocyte-specific promoters, megasporangium-specific promoter-0, integument-specific promoters, stigma-specific promoters, and style-specific promoters. In one example, floral-specific promoters include an embryo-specific promoter or a late embryo-specific promoter, such as the late embryo specific promoter of DNH 1 or the HVA1 promoter, the GLB1 promoter from corn, and any of the Zein promoters (Z27). In another example, floral-specific promoters include the FLO/LFY promoter from switchgrass.

The determination of whether a sequence operates to confer floral specific expression in a particular system (taking into account the plant species into which the construct is being introduced, the level of expression required, etc.), is preformed using known methods, such as operably linking the promoter to a marker gene (e.g. GUS, and GFP), introducing such constructs into plants and then determining the level of expression of the marker gene in floral and other plant tissues. Sub-regions which confer only or predominantly floral expression, are considered to contain the necessary elements to confer floral specific expression.

Functional deletion: A gene is functionally deleted when the function of the gene or gene product is reduced or eliminated. For example, anti-sense molecules can be used to functionally delete a gene. In another example, a cell or tissue is functionally deleted when the function of the cell or tissue is reduced or eliminated. For example, cytotoxic genes, such as barnase, can be used to functionally delete floral-specific cells, such as the tapetum, thereby resulting in sterility of the plant.

Functionally equivalent: Nucleic acid sequence alterations in a vector that yield the same results described herein. Such sequence alterations can include, but are not limited to, conservative substitutions, deletions, mutations, frameshifts, and insertions. For example, in a nucleic acid including a barnase sequence that is cytotoxic, a functionally equivalent barnase sequence may differ from the exact barnase sequences disclosed herein, but maintains its cytotoxic activity. Methods for determining such activity are disclosed herein.

Gene of interest: (GOI) Any gene, or combination of functional nucleic acid sequences (such as comprised in plant expression cassettes such as with a promoter, coding sequence and termination sequence) in plants that may result in a desired phenotype.

Isolated: An “isolated” biological component (such as a nucleic acid or protein) has been substantially separated, produced apart from, or purified away from other biological components in the cell of the organism in which the component naturally occurs, i.e., other chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids, proteins and peptides.

Nucleic acid: A deoxyfibonucleotide or ribonucleotide polymer in either single or double stranded form, and unless otherwise limited, encompasses known analogues of natural nucleotides that hybridize to nucleic acids in a manner similar to naturally occurring nucleotides.

Oligonucleotide: A linear polynucleotide (such as DNA or RNA) sequence of at least 9 nucleotides, for example at least 15, 18, 24, 25, 27, 30, 50, 100 or even 200 nucleotides long.

ORF (open reading frame): A series of nucleotide triplets (codons) coding for amino acids without any termination codons. These sequences are usually translatable into a peptide.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

Peptide: A chain of amino acids of which is at least 4 amino acids in length. In one example, a peptide is from about 4 to about 30 amino acids in length, for example about 8 to about 25 amino acids in length, such as from about 9 to about 15 amino acids in length, for example about 9-10 amino acids in length.

Perennial: A plant which grows to floral maturity for three seasons or more. Whereas annual plants sprout from seeds, grow, flower, set seed and senesce in one growing season, perennial plants persist for several growing seasons. The persistent seasonal flowering of perennial plants may also, but not necessarily, include light and temperature requirements that result in vernalization. Examples include, but are not limited to: certain grasses, such as turfgrass, forage grass or ornamental grasses; trees, such as fruit and nut crop trees (for example bananas and papayas), forest and ornamental trees, rubber plants, and shrubs; grapes; roses; and wild rice.

Pollen-specific gene: A DNA sequence that directs a higher level of transcription of an associated gene in microspores and/or pollen (i.e., after meiosis) relative to the other tissues of the plant. Examples include, but are not limited to: pollen-specific promoters LAT52, LAT56, and LAT59 from tomato (GenBank Accession Nos. BG642507, X56487 and X56488), rice pollen specific gene promoter PSI (GenBank Accession No. Z16402), and pollen specific promoter from corn (GenBank Accession No. BD136635 and BD136636).

Pollen-specific promoter: A gene sequence that is primarily expressed in pollen relative to the other cells of the plant. Includes any pollen-specific gene whose malfunction or functional deletion results in male-sterility. Examples include, but are not limited to: LAT52, LAT56, and LAT59 from tomato (GenBank Accession Nos. BG642507, X56487 and X56488), PSI (GenBank Accession No. Z16402), and pollen specific gene from corn (GenBank Accession No. BD136635 and BD136636).

Promoter: An array of nucleic acid control sequences that directs transcription of a nucleic acid. A promoter includes necessary nucleic acid sequences near the start site of transcription, such as, in the case of a polymerase II type promoter, a TATA element. A promoter also optionally includes distal enhancer or repressor elements that can be located as much as several thousand base pairs from the start site of transcription. Both constitutive and inducible promoters are included.

Specific, non-limiting examples of promoters that can be used to practice the disclosed methods include, but are not limited to, a floral-specific promoter, constitutive promoters, as well as inducible promoters for example a heat shock promoter, a glucocorticoid promoter, and a chemically inducible promoter. Promoters produced by recombinant DNA or synthetic techniques may also be used. A polynucleotide encoding a protein can be inserted into an expression vector that contains a promoter sequence that facilitates the efficient transcription of the inserted genetic sequence of the host. In one example, an expression vector contains an origin of replication, a promoter, as well as specific nucleic acid sequences that allow phenotypic selection of the transformed cells.

Recombinase: A protein which catalyses recombination of recombining sites. Non-limiting examples of recombinases include CRE, FLP, Tn3 recombinase, transposon gamma/delta, and transposon mariner.

The cre and Flp proteins belong to the lambda/integrase family of DNA recombinases. The cre and Flp recombinases are similar in the types of reactions they carry out, the structure of their target sites, and their mechanism of recombination. For instance, the recombination event is independent of replication and exogenous energy sources such as ATP, and functions on both supercoiled and linear DNA templates.

Recombinases exert their effects by promoting recombination between two of their recombining sites. In the case of cre, the recombining site is a Lox site, and in the case of Flp the recombining site is a Frt site. Similar sites are found in transposon gamma/delta, TN3, and transposon mariner. These recombining sites include inverted palindromes separated by an asymmetric sequence. Recombination between target sites arranged in parallel (so-called “direct repeats”) on the same linear DNA molecule results in excision of the intervening DNA sequence as a circular molecule. Recombination between direct repeats on a circular DNA molecule excises the intervening DNA and generates two circular molecules. The cre/Lox and flp/frt recombination systems have been used for a wide array of purposes such as site-specific integration into plant, insect, bacterial, yeast and mammalian chromosomes has been reported (Sauer et al., Prvc. Natl. Acad. Sci. USA, 85:5166-70, 1988. Positive and negative strategies for selecting or screening recombinants are known.

Recombining site: A nucleic acid sequence that includes inverted palindromes separated by an asymmetric sequence (such as a transgene) at which a site-specific recombination reaction can occur. Examples include, but are not limited to, Lox, Frt (consists of two inverted 13-base-pair (bp) repeats and an 8-bp spacer that together comprise the minimal Frt site, plus an additional 13-hp repeat which may augment reactivity of the minimal substrate, TN3, mariner, and a gamma/delta transposon.

Recombinant: A recombinant nucleic acid is one that has a sequence that is not naturally occurring or has a sequence that is made by an artificial combination of two otherwise separated segments of sequence. This artificial combination can be accomplished, for example, by chemical synthesis or by the artificial manipulation of isolated segments of nucleic acids, e.g., by genetic engineering techniques. Similarly, a recombinant protein is one encoded for by a recombinant nucleic acid molecule.

Selectable marker: A nucleic acid sequence that confers a selectable phenotype, such as in plant cells, that facilitates identification of cells containing the nucleic acid sequence. Transgenic plants expressing a selectable marker can be screened for transmission of the gene(s) of interest. Examples include, but are not limited to: genes that confer resistance to toxic chemicals (e.g. ampicillin, spectinomycin, streptomycin, kanamycin, geneticin, hygromycin, glyphosate or tetracycline resistance, as well as bar and pat genes which confer herbicide resistance), complement a nutritional deficiency (e.g., uracil, histidine, leucine), or impart a visually distinguishing characteristic (e.g., color changes or fluorescence, such as 13-gal).

Tapetum-specific gene: A gene sequence that is primarily expressed in the tapetum relative to the other tissues of the plant. Includes any tapetum cell-specific gene whose malfunction results in male-sterility. Examples include, but are not limited to: TA29 and TA13, pca55, pE1 and pT72, Bcp1 from Brassica and Arabidopsis (GenBank Accession Nos. X68209 and X68211), A9 from Brassicaceae (GenBank Accession No. A26204), and TAZ1, a tapetum-specific zinc finger gene from petunia (GenBank Accession No. AB063169).

Tapetum-specific promoter: A DNA sequence that directs a higher level of transcription of an associated gene in tapetal tissue relative to the other tissues of the plant. Tapetum is nutritive tissue required for pollen development. Examples include, but are not limited to the promoters associated with the genes listed under tapetum-specific genes.

Transduced and transformed: A virus or vector “transduces” or transfects” a cell when it transfers nucleic acid into the cell. A cell is “transformed” by a nucleic acid transduced into the cell when the DNA becomes stably replicated by the cell, either by incorporation of the nucleic acid into the cellular genome, or by episomal replication. As used herein, the term transformation encompasses all techniques by which a nucleic acid molecule can be introduced into such a cell. Examples include, but are not limited to, transfection with viral vectors, transformation with plasmid vectors, electroporation, lipofection, Agrobacterium-mediated transfer, direct DNA uptake, and microprojectile bombardment.

Transgene: An exogenous nucleic acid sequence. In one example, a transgene is a gene sequence, for example a sequence that encodes a cytotoxic polypeptide. In yet another example, the transgene is an antisense nucleotide, wherein expression of the antisense nucleotide inhibits expression of a target nucleic acid sequence. A transgene can contain native regulatory sequences operably linked to the transgene (e.g. the wild-type promoter, found operably linked to the gene in a wild-type cell). Alternatively, a heterologous promoter can be operably linked to the transgene.

Transgenic Cell: Transformed cells that contain a transgene, which may or may not be native to the cell.

Vector: A nucleic acid molecule as introduced into a cell, thereby producing a transformed cell. A vector can include nucleic acid sequences that permit it to replicate in the host cell, such as an origin of replication. Examples include, but are not limited to a plasmid, cosmid, bacteriophage, or virus that carries exogenous DNA into a cell. A vector can also include one or more cytotoxic genes, antisense molecules, and/or selectable marker genes and other genetic elements known in the art. A vector can transduce, transform or infect a cell, thereby causing the cell to express the nucleic acids and/or proteins encoded by the vector. A vector optionally includes materials to aid in achieving entry of the nucleic acid into the cell, such as a, liposome, protein coating or the like.

Zinc-finger nucleases (ZFNs): ZFNs are synthetic endo-restriction enzymes generated by fusing a zinc finger DNA-binding domain(s) to a DNA-cleavage domain.

### Sequence Listing

The nucleic acid sequences listed in the accompanying sequence listing are shown using standard letter abbreviations for nucleotide bases. Only one strand of each nucleic acid sequence is shown, but the complementary strand is understood as included by any reference to the displayed strand.

SEQ ID NO: 1 is a nucleic acid sequence of a corn ovule-specific gene;

SEQ ID NO: 2 is a nucleic acid sequence of a corn female inflorescence developmentally-specifically expressed gene;

SEQ ID NO: 3 is a nucleic acid sequence of a corn tapetum-specific gene; and

SEQ ID NO: 4 is a nucleic acid sequence of a corn pollen-specific gene.

### Review of Methods to Create Male and Female Sterile Lines

### Gene Confinement Methods

Previous methods for gene confinement have been developed including; 1. Non-biological methods: physical, spatial, mechanical and temporal methods; and, 2. Biocontainment methods: male sterility, cytoplasmic male sterility and chloroplast transformation technologies; seed-based gene confinement; the gene deletor system; and various total sterility concepts. These approaches are discussed in the forthcoming section. The current invention is differentiated by the ability to recover stable deletion mutants that can be hybridized to create sterile progeny.

### Non-Biological Methods: Physical, Spatial, Mechanical and Temporal Methods

Conceivably transgenic plants can be confined spatially and temporally using non-biological methods. Physical containment includes specific cases such as production of plant-manufactured pharmaceuticals in greenhouses, underground facilities, inside buildings, or in cultivation areas unique to a specific crop, such as growing rice in Kansas. Many transgenic crops will likely be so extensively widespread that physical confinement is not feasible. Mechanical control of flowering would be one strategy to contain transgenes in feedsocks; e.g., pollen and seed production could be prevented by mowing perennial grasses. However, frequent mowing would be costly and subject to human error, and thus, not feasible for bioenergy feedstocks.

### Biocontainment Methods

### Male Sterility

The primary route of gene flow in transgenic plants will be through pollen, thus prevention of viable pollen production represents a potential biocontainment strategy as well as important to plant breeding. Indeed, there has been much research on engineering male-sterility for hybrid plant production, biocontainment, and other purposes. One target for male-sterility is ablation of the tapetum, the innermost layer of the anther wall that surrounds the pollen sac, which is needed for pollen development. A variety of anther and tapetum-specific genes have been identified that are involved in normal pollen development in many plant species, including maize, rice, tomato, Brassica campestris, and Arabidopsis thaliana. Selective ablation of tapetal cells by cell-specific expression of nuclear genes encoding cytotoxic molecules or an antisense gene essential for pollen development blocks pollen development, giving rise to stable male sterility.

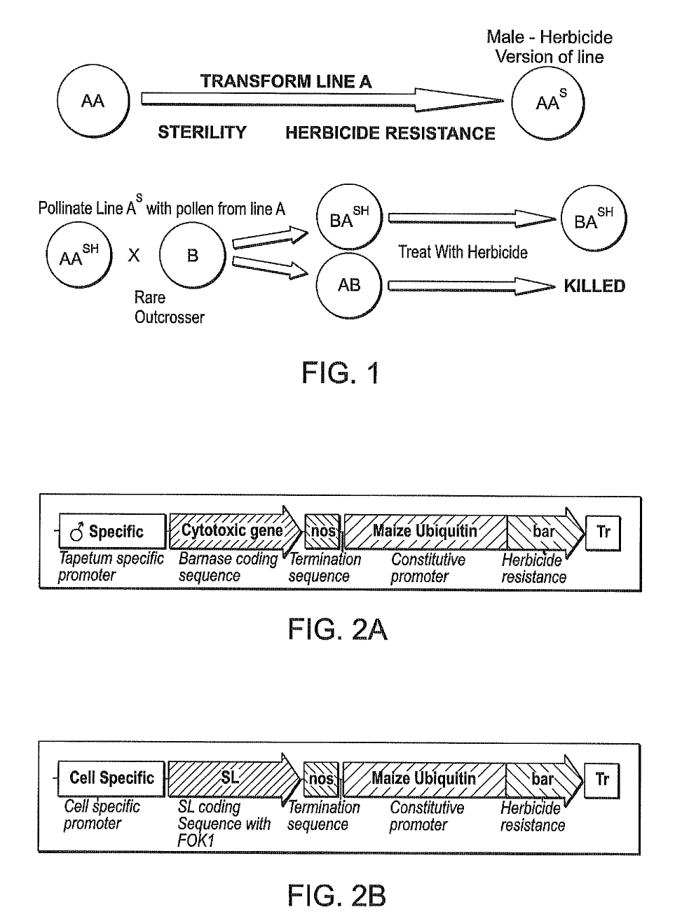

The process shown in FIG. 1 is directed to the development of hybrid plant systems based on sterility. Male sterile hybrid plants were crossed with female patents to recover herbicide resistant plants which are crossed out using MAB to produce non-GMO varieties. More specifically, to induce male sterility in turfgrass, the 1.2-kb rice rts gene regulatory fragment, TAP was fused with two different genes (See FIG. 1). One was the antisense of rice rts gene that is predominantly expressed in tapetum cells during meiosis. Another gene was the Bacillus amyloliquefaciens ribonuclease gene, barnase, which ablates tapetal cells by destruction of RNA. Both of these approaches have been shown to be effective in various plant species. Field performance of these plants resulted in the recovery of three herbicide resistant plants from over 10⁵ tested wild type seeds indicating low leakage of the system. Therefore, nuclear male sterility, resulting in the lack of viable pollen grains, when linked to the genes of agronomic interest provides an important tool to study effective mechanisms for interrupting gene flow. In addition, male sterile lines will provide important breeding tools.

### Cytoplasmic Male Sterility and Chloroplast Transformation Technologies

Cytoplasmic male sterility (CMS) and chloroplast transformation also offer choices for controlling gene flow between dedicated energy crops and their wild relatives. CMS is caused by mutations in the genomes of either the chloroplast or the mitochondria and are exclusively maternally inherited in many plant species. In crop plants, nuclear genes which restore fertility (RD have been widely applied for creating hybrids. Consequently, the development of CMS systems for dedicated energy crops would be useful for gene confinement as well as providing valuable breeding tools for these crops. However, the current status of breeding efforts for these crops does not yet include these tools. An attractive option would be to genetically engineer a CMS-associated mitochondrial gene for stable nuclear expression such that pollen production would be disrupted.

The first engineered cytoplasmic male sterility system in plants was accomplished by expression of β-kethiolase by stable integration of the phaA gene via the chloroplast genom. Prior attempts to express the phaA gene in transgenic plants were unsuccessful. The phaA gene was efficiently transcribed in all tissue types including leaves, flowers and anthers. Coomassie-stained gel and western blots confirmed hyper-expression of β-ketothiolase in leaves and anthers, with proportionately high levels of enzyme activity. The transgenic lines were normal except for the male sterile phenotype, lacking pollen. Scanning electron microscopy revealed a collapsed morphology of the pollen grains. Floral developmental studies revealed that transgenic lines showed an accelerated pattern of anther development, affecting their maturation and resulted in aberrant tissue patterns. Abnormal thickening of the outer wall, enlarged endothecium and vacuolation affected pollen grains and resulted in the irregular shape or collapsed phenotype. This method offers yet another tool for transgene containment and provides an expedient mechanism for F1 hybrid seed production.

Integration of transgenes into the chloroplast genome is an approach to accomplish both transgene biocontainment and high levels of transgene expression without the possibilities for gene silencing or position effects. Maternal inheritance of genetically modified chloroplast genomes and the absence of any reproductive structures when foreign proteins expressed in leaves are harvested, offer efficient transgene containment via pollen or seeds and facilitates their safe production in the field. Two recent studies point out efficient control of maternal inheritance of transgenes in transplastomic tobacco. Ruf S, Karcher D, Bock R: Determining the transgene containment level provided by chloroplast transformation. Proc. Natl. Acad. Sci. USA 104, 6998-7002 (2007) set up a stringent selection system for paternal transmission by using male sterile maternal parents and transplastomic pollen donors conferring plastid specific antibiotic resistance and green fluorescence for visual screening. This selection system identified six among 2.1 million seedlings screened (frequency of 2.86×10⁻⁶) that showed paternal transmission of transgenes and the authors concluded that plastid transformation provided an effective tool to increase biosafety of GM crops. Therefore, transplastomic plants producing human therapeutic proteins have been already tested in the field after obtaining USDA-APHIS approval.

While not offering absolute transgene containment, confining transgenes within chloroplast genomes will greatly limit the passage of transgenes via pollen and therefore to other crops or relatives via outcrossing. However, if transgene products are harvested from leaves before the appearance of any reproductive structures, absolute transgene containment via pollen or seeds are possible. The major technical challenge to this possible containment strategy is to get the transgene into every chloroplast (homoplasmy) in each cell. However, only three rounds of selection on regeneration media are typically required to reach homoplasmy in tobacco. Southern blots and PCR are used to measure if any wildtype copies are present and homoplasmic lines can be identified and increased. Since chloroplasts are prokaryotic compartments, they lack the silencing machinery found within the cytoplasm of eukaryotic cells. Each plant cell contains 50-100 chloroplasts and each chloroplast contains ˜100 copies of its genome, so it is possible to introduce 20,000 copies of the transgene per cell. Transgenes have been stably integrated and expressed via the tobacco chloroplast genome to confer important agronomic traits including herbicide, insect, and disease resistance, drought and salt tolerance, cytoplasmic male sterility or phytoremediation. Chloroplast genomes of several crop species including cotton, soybean, carrot, sugarbeet, cauliflower, cabbage, oilseed rape, poplar, potato, tomato, tobacco, lettuce and other crops have been transformed. Twenty four vaccine antigens against 16 different diseases and twelve biopharmaceuticals including insulin and interferon have been expressed in tobacco chloroplasts and many are fully functional. Complete chloroplast genome sequences of more than thirty crop species have been determined recently, facilitating rapid advancement in this field. Chloroplast transformation in cereal crops is feasible but it should be developed in dedicated energy crops (e.g., perennial grasses, sorghum, maize, etc.).

Biofuel production from lignocellulosic materials is limited by the lack of technology to efficiently and economically release fermentable sugars from the complex multi-polymeric raw materials. Therefore, mixtures of enzymes containing endoglucanases, exoglucanase, pectate lyases, cutinase, swollenin, xylanase, acetyl xylan esterase, beta glucosidase and lipase genes from bacteria or fungi have been expressed in tobacco chloroplasts. Homoplasmic transplastomic lines showed normal phenotype and were fertile. Chloroplast-derived crude-extract enzyme cocktails yielded more (up to 3,625%) glucose from filter paper, pine wood or citrus peel than commercial cocktails and 1000-3000 fold cheaper than recombinant commercial enzymes. Although individual enzymes have been expressed in plants before, this is the first report of production of recombinant enzyme cocktails from transgenic plants. Transgene containment is a serious concern in transgenic plants expressing cell wall hydrolyzing enzymes via the nuclear genome because of their toxicity to out-crossing crops or weeds and therefore biological containment via maternal inheritance or their harvest before appearance of any reproductive structures is essential for biocontainment.

### Seed-Based Gene Confinement

Seed-based biocontainment relies on the use of genetic use restriction technologies (GURTs). Even though this apparently biased terminology emphasizes only the proprietary protection issues of corporate interests, perhaps the most impactful use of GURTs is related to transgene biocontainment. There are two major classes of GURTs: V-GURTs (varietal-level GURTs) and T-GURTS (trait-specific GURTs) which correspond to growth stage that trigger a genetic switch for containment. When triggered, V-GURT systems prevent the propagation of the crop and its associated genetic technology without the purchase of new seed. V-GURTs allow for normal growth and full development of the desired seed; however the progeny seed, if planted, will not germinate. Gene containment is achieved by the inability of the plants that contain the activated V-GURT mechanism to produce viable progeny either through the pollen or via seed. T-GURT systems regulate trait expression making the value-added trait (transgene) available only if the farmer triggers the genetic switch mechanism. Plant function is normal, but when a particular engineered trait is needed in a farmer's field, a specific triggering chemical purchased from the technology provider is applied to activate transgenes expressing a desired characteristic (e.g., insect resistance). The technology would presumably only be paid for and activated when needed. Transgene biocontainment would be achieved by the inability of the plants to express the transgenic trait in the absence of the activating chemical that is not indigenous in the environment.

One of the major issues raised in objection to the use of V-GURTs is the possible impact on seed viability in compatible non-transgenic or T-GURT crops in neighboring fields as a result of the spread of pollen from a V-GURT crop. V-GURTS are currently time designed for use in crops that preferentially self-pollinate rather than outcross, e.g., cotton, soybean and wheat. In such cases negative effects on neighboring fields would be very restricted and would not be detectable above the background of normal germination rates for field grown crops. V-GURTs targeted for crops that readily outcross would have to contain design elements for the removal of transgenes during microsporogenesis so as to prevent transgene escape via pollen dispersal. A similar concern has been posed in regards to the possibility that pollen from V-GURT plants could prevent germination of seeds in neighboring wild species and thus reduce their long-term viability in the native habitat. Obviously preventing the germination of hybrid seed developed from pollen outflow from a crop to a wild species is a premium outcome in the desire to contain transgenes in the environment, but it would be problematic if the long term viability of a wild species is affected. In realistic terms this is an unlikely scenario because such an outcome would require that the wild species was completely compatible with the crop containing the V-GURT and that non-V-GURT pollen was absent from the environment; i.e., no genetic barriers between types. Most crops do not have relatives that are sexually compatible in agricultural areas and hybridization is rare. In cases where there is a measure of compatibility and a problem exists, a change in the design of the V-GURT may be warranted.

V-GURTs have also been criticized for their supposed potential for socio-economic impacts on agriculture in developing countries. The non-germinability of GeneSafe seeds and the resultant need to purchase new seed for the planting of a new crop has been suggested to be an unfair economic burden on small farmers especially those engaged in subsistence farming. Although it is true that farmers would be required to purchase new seed every year one has to bear in mind that GeneSafe and other V-GURT technologies alone have no value and would only be in a crop in conjunction with a valuable or advantageous transgenic trait; i.e., V-GURTs and the trait are linked. Indeed, GeneSafe technologies would allow subsistence farmers access to superior traits that would have the potential to increase and ensure yields and thus deliver term from the vagaries of the environment within which they practice, perhaps to the point of enabling the establishment of a production level operation.

Environmental concerns have been raised that the method used to prevent the germination of activated V-GURT seeds could harm other organisms. The currently used gene products disrupt seed metabolism; they are not toxic to animals and occur naturally in plants and microbes that are normally consumed in animal diets. Similarly, the chemical seed treatment used to activate the V-GURT during stand establishment would have to be, by necessity, environmentally friendly or neutral. The use of tetracycline described in the GeneSafe prototype was never targeted for commercial use in the field.

Transgenic seedless fruits (although not a complete gene containment technology) described by Tomes D T, Huang B, Miller P D, Genetic constructs and methods for producing fruits with U.S. Pat. No. 5,773,697 (1998); and the GeneSafe technologies of Oliver M J, Quiseberry J E, Trolinder N, Glover L, Keim D L: Control of plant gene expression, U.S. Pat. No. 5,723,765 (1998): Oliver M J, Quisenberry J E, Trolinder N, Glover L, Keim D L: Control of plant gene expression, U.S. Pat. No. 5,977,441 (1999): and Oliver M J, Quisenberry J E, Trolinder N, Glover L, Keim D L: are all V-GURTs designed to prevent gene out-flow from transgenic plants via seeds. The basic strategy outlined in these patents is to control the activation of a “germination disruption gene” such that its expression prevents establishment of the next generation of a crop that bears a value-added or production-benefit transgene. The gene activation is timed such that the transgene is available in an uncontained environment such as a farmer's field, and it is only after a crop is produced that the activated germination disruption gene is expressed and effective. The mechanism is also designed such that pollen from a plant that contains the activated germination disruption gene fertilizes an ovule and generates a non-germinable seed. Although this is desired for total gene containment, this could be problematic in an open pollination scenario. The GeneSafe mechanisms described here were designed for crops that reproduce under restricted or mainly closed pollination. The three elements needed for GeneSafe are 1) a promoter that responds to a specific exogenous stimulus; 2) A site-specific recombinase to remove a physical block; and 3) a seed-specific promoter that is only active late in seed development. These elements were used to generate two genetic systems (basic systems from which refinements can be added), one based on a repressible promoter mechanism that is relieved by exposure to an activator and the other, a more simple system based on a chemically inducible promoter. These two mechanisms were originally designed for use in GM cotton as a technology protection system.

At the present time, the repressible GeneSafe technology has been developed in both cotton and tobacco to varying degrees, tobacco being the most advanced. Germination tests of seed derived from selfing seedling activated (tetracycline treated) dual hemizygous plants that exhibit precise excision in vegetative cells of the plants did not generate the expected 3:1 ratio of non-germinable to germinable seed (assuming successful activation of CRE in all germline cells of the parental lines). In fact in only a few cases were germination percentages reduced. However, PCR analysis of the seeds used in the germination tests revealed that all were either heterozygous for the excision phenotype or homozygous for the intact module; no seeds homozygous for the excision event were detected (360 seed lots tested so far, Oliver et al. unpublished data). The implication is that seeds that contain two copies of the excision event do not develop to maturity in tobacco pods of plants derived from tetracycline-treated seeds. This would further imply that the timing of expression of the protein synthesis inhibitor driven by the cotton LEA promoter in tobacco does not mimic that seen in cotton, i.e., it occurs prior to the maturation phase of seed development, and that the level of expression of the protein synthesis inhibitors suffices to affect viability when only one copy of the gene is present. Research is ongoing in this pilot study.

### Gene Deletor System

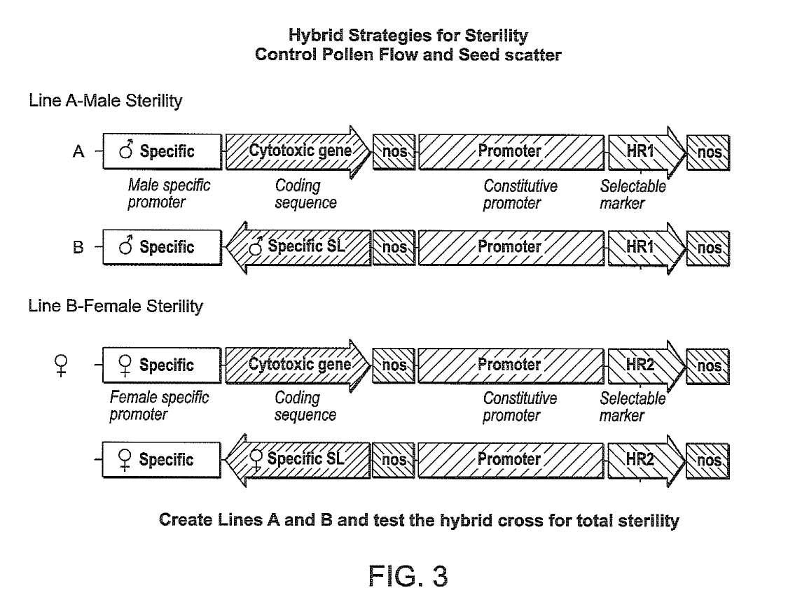

FIG. 3 is directed to hybrid strategies for sterility constructs, including both male and female sterility constructs and include different promoters. A highly efficient system to delete all transgenes from pollen or both pollen and seed has been developed. In this method transgenic cassettes are effectively excised using components from both FLP/FRT and CRE/loxP recombination systems. When loxP-FRT fusion sequences (86 bp) were used as recognition sites, simultaneous expression of both FLP and CRE reduced the average excision efficiency, but the expression of either FLP or Cre alone increased the average excision efficiency. When three different gene promoter sequences were used to control the expression of the FLP or Cre gene, transgenic tobacco events with 100% efficiency in transgene deletion from pollen, or both pollen and seed were observed based on analysis of more than 25,000 T1 progeny. The deletion of all functional transgenes from pollen, or both pollen and seed was confirmed using three different techniques: histochemical GUS assays, Southern blot analysis and PCR. These studies were conducted in tobacco under greenhouse conditions and have not yet been field tested. The gene deletor system, which can produce ‘non-transgenic’ pollen and/or seed from transgenic plants, may provide a useful biocontainment tool for transgenic crops and perennials, and may be applicable for vegetatively propagated biofuel plants. If a conditionally-inducible gene promoter, such as a chemically- or high-temperature-inducible or postharvest-stage active promoter were used to control recombinase expression, all functional transgenes could be deleted throughout the plant on application of the inducer or after harvesting.

### Total Sterility

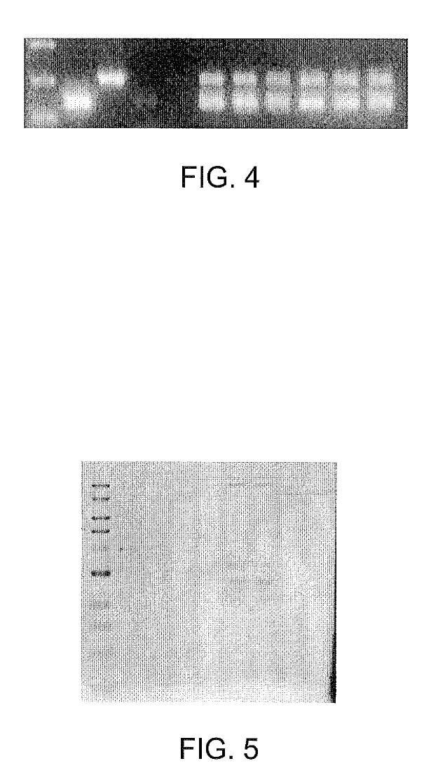

H. Luo, A. Kausch, J. Chandlee and M. Oliver (2005, unpublished) proposed mechanism to eliminate all possibility for gene transfer in species that are primarily grown for their green biomass, in particular turf grasses. See FIG. 4, which shows a PCR amplification for bar & barnase genes Lane 1: PCR ladder; lane 2: bar primers+(plasmid); lane 3: barnase primers+(plasmid); lanes 4-5: negative controls; lanes 6-11: bar & barnase amplification from 6 individual transgenic event. The strategy hinges on the prevention of flowering using a site-specific recombinase (in this case the FLP/FRT system from yeast) to activate a gene designed to down-regulate a gene critical in the initiation of floral development. The targeted gene for down-regulation is FLORICAULA/LEAFY, which triggers the vegetative to reproductive developmental transition of meristems. The mechanism operates by establishing a transgenic line homozygous for both the transgene of interest and a genetic construct containing the following linked elements: a constitutive plant promoter—an FRT site (recognition site for FLP)—a blocking sequence—an FRT site—RNAi or antisense construction for FLORICAULA/LEAFY. In the final seed production cycle homozygous plants are crossed to plants homozygous for a constitutively expressed FLP gene to produce hybrid seed. When grown the hybrid seeds will generate plants that constitutively express FLP resulting in the excision of the blocking sequence contained in the initial construct. This will activate the constitutive expression of the RNAi or antisense construction for FLORICAULA/LEAFY. This in turn will down regulate the expression of the endogenous FLORICAULA/LEAFY genes rendering the plant incapable of producing flowers. The vegetative growth habit of the hybrid retains its commercial application but is incapable of transferring transgenes to neighboring grasses or weedy relatives. This is in effect a hybrid total gene containment system. Variation on this scheme is possible to include selection of the outcome using two herbicide resistance genes insuring the hybrid seed. The results are illustrated in the PCR amplification of FIG. 4 illustrating the male sterility with barnase and the Southern blot analysis of FIG. 5 shows the transgenic switchgrass plants. In particular, FIG. 5 shows a sample Southern blot analysis of transgenic switchgrass plants, including lane 1: molecular wt markers; lane 2: positive control from plasmid DNA; lane 3: negative control: switchgrass wild-type DNA; and lanes 4-9: Southern analysis-3 independent events.

This application is directed to the disruption of fertility in flowering plants. Gene flow between transgenic plants and wild and non-transgenic relatives is widely understood as a major obstacle to genetic improvement of perennial plants. Synthetic Lethality (SL) of male and female reproduction offers a solution to both breeding and gene flow issues. A solution to this problem has been exemplified with the development of improved perennial plants, such as switchgrass, for the biofuels industry and a method for the solution to the problem of gene confinement. In addition, this method provides a controlled sex determination in plants provided a unique breeding advantage for cereal crops and other grasses.

Thus, provided herein is a unique approach to gene containment by using male and female sterile lines to create sterile hybrid plants to control gene flow in genetically modified plans and facilitate breeding.

Methods for generating male and female sterile lines have been developed using three methods with SLs including 1) male and female specific cell ablation that results in sterile hybrids; 2) SL directed cell ablation for reproductive specific male and female genes that result in male and female sterile lines that can be used for breeding; and 3) creation of stable knockout mutations in genes required for fertility whereby using these lines from either method or in concert in crosses will create hybrid progeny that will be completely sterile. In addition, these approaches will create populations significant to breeding efforts in these crops and other plants.

Below are methods for producing male and female sterile lines of plants resulting in completely sterile progeny when crossed to produce hybrids. First, methods are disclosed to make male sterile lines by using targeted sequences specific for male reproductive structure development or maintenance, and by using zinc finger nuclease technology or other SL technologies together with transgenics, to create knockout mutations to generate male sterile lines. Second, methods are disclosed to make female sterile lines by targeting sequences specific for female reproductive structure development or maintenance, and by also using zinc finger nuclease technology together with transgenics and other SL technologies, creating male sterile lines. Thirdly, methods are disclosed for creating targeted insertions with a gene of interest (GOI) into regions that result in either male or female sterility using the first two described methods. Fourth, methods are disclosed for the ablation of seeds in the hybrid plant. Lastly, methods are described for the combination of male and female sterile lines for the purpose of gene confinement and for breeding. They also increase the heterozygocity.

In the first example, the method includes contacting a plant, or plant cell, with a vector, wherein the vector includes a construct to express zinc finger nucleases specific and other targeted ablations to male female floral specific gene(s), including, but not restricted to, the developing filament, anther, microsporcytes, pollen, female parts and operably linked to a plant promoter. The plant promoter may be operably linked to a tissue specific promoter or can be constitutively expressed. The production of transgenics may or may not include the use of a selectable marker gene, but the preferred example is using selection. Expression of this vector results in the production of a male (pollen) deficient plant, thereby producing a producing a plant having reduced or no functional male gametes.

In the second example, the method includes contacting a plant or plant cell, with a vector, wherein the vector includes a construct to express zinc finger nucleases specific or other knock out methods to eliminate the function of female floral specific gene(s), including, but not restricted to, the developing style, stigma, ovule, integuments megagametophyte, endosperm and eggs, operably linked to a plant promoter. The plant promoter may be operably linked to a tissue specific promoter or can be constitutively expressed. The production of transgenics may or may not include the use of a selectable marker gene, but the preferred example is using selection. Expression of this vector results in the production of a female (seed) deficient plant, thereby producing a producing a plant having reduced or no functional male gametes.

In the third example, the method includes contacting a plant, or plant cell, with a vector, wherein the vector includes a construct to express zinc finger nucleases specific to either male or female floral specific gene(s), including those described above, operably linked to a plant promoter and including a gene of interest (GOI) targeted to disrupt the floral specific genes. The plant promoter may be operably linked to a tissue specific promoter or can be constitutively expressed. The production of transgenics may or may not include the use of a selectable marker gene, but the preferred example is using selection. The preferred example would use an herbicide resistance marker in the male, but the female may also be used as well as using two compatible marker to create a doubly selectable hybrid. Expression of this vector results in the production of a seed deficient plant, thereby producing a producing a plant having reduced or no progeny.

In the third example, the method includes contacting a plant, or plant cell, with a vector, wherein the vector includes a construct to express zinc finger nucleases specific or other knockout technique to either male or female floral specific gene(s), including those described above, operably linked to a plant promoter and utilizing site specific recombination to facilitate expression of the zinc finger nucleases in the F1 population resulting in sterile seeds and or total vegetative growth habit. Methods are disclosed for creating hybrid seeds and totally vegetative plants.

For example, the vector can be transfected into cells of the plant that result in the recovery of a stable transgenic plant capable of Mendellian segregation for either the transgene, the targeted knockout mutation or both. Examples of plants that can be used include, but are not limited to, corn, rice, switchgrass, Atlantic Coastal Panic Grass, Big Blue stem, poplar trees, sugar cane, and jatropha, Paulownia.

The plant having either male, female, or embryo sterility can have one or more desirable traits, or as two or more desirable traits, such as resistance to insects and other pests and disease-causing agents; tolerances to herbicides; post harvest activation of cellulase or other enzymes related to biofuel production methods; increased starch production; enhanced stability or yield; decreased lignin, increased cellulose; environmental tolerances; ease of hydrolysis, and ethanol production enhancements. The desirable traits can be linked to the gene which results in herbicide resistance or other selection. In one example, the desired trait is due to the presence of a transgene(s) in the plant. In another or additional example, the desired trait is obtained through conventional breeding. In addition the trait can be introduced through breeding to deliver a GOI which can then be sequestered in the sterile hybrid plant. In this way additional genes can be added into a sterility platform. In another example, traits can maintained through vegetative propagation in totally sterile plant hybrids In this example the trait for is produced as the outcome of a cross between two parents each with one component of the floral deficiency system. The unlinked trait will always remain in the sterile background preventing the possibility of escape due to segregation of recombination in future generations. These parents can also carry one or more additional desirable traits.

Also disclosed are methods for producing a controlled total vegetative growth phenotype in perennial plants, as well as perennial plants produced by such methods, such as a male-deficient and/or female deficient perennial plant vegetatively propagated asexual plants, and seeds produced by parents of the plants that when crossed will produce an asexual or floral-deficient plant. In one example, the method includes crossing a first male sterile plant having one or more desirable traits, such as two or more desirable traits, with second female sterile plant having one or more desirable traits, such as two or more desirable traits. The first male sterile plant includes a SL induced mutation, wherein the mutation occurs in a male floral-specific gene or sequence required for fertility and the vector includes a construct specific and operably linked to a blocking sequence, such as a selectable marker, and recombining site sequences flanking the blocking sequence. In addition, the construct includes a cytotoxic sequence, which is downstream to the promoter and the blocking sequence, and is in a position such that its expression is activated by the floral-specific promoter in the presence of a recombinase, which results in recombination at the recombining site sequences and removal of the blocking sequence. The second female sterile plant includes another vector which includes a promoter operably linked to a recombinase. The promoter can be a constitutive promoter or an inducible promoter. If an inducible promoter is used, the second plant is contacted with an inducing agent, before, during, or after crossing the first and second fertile plant. The inducing agent activates the inducible promoter, thereby permitting recombinase expression. If a constitutive promoter is used, the promoter will drive recombinase expression in the absence of an inducing agent. The expressed recombinase protein interacts with the recombining sites of the other vector, resulting in recombination, removal of the blocking sequence such that the floral-specific promoter is now operably linked to the cytotoxin, thereby driving expression of the cytotoxin in floral-specific tissues. The resulting progeny of such a cross are asexual or floral-deficient. In one example, the vector included in the second sterile plant which also includes a promoter operably linked to a blocking sequence. These vectors can be stably integrated into the genome of the plant.

Many floral-specific genes and promoters can be used as targets to practice the disclosed methods, including variants thereof that are functionally equivalent and confer gene express in or predominantly in floral tissues. Particular examples include, but are not limited to: floral-specific promoters and genes, such as the FLORICA ULA/LEAFY homolog, anther-specific promoters and genes, pollen-specific promoters and genes, ovule-specific promoters and genes, megasporocyte-specific promoters and genes, megasporangium-specific promoters and genes, integument-specific promoters and genes, stigma-specific promoters and genes, and style-specific promoters and genes. In one example, floral-specific promoters and genes include an embryo-specific promoter and genes or a late embryo-specific promoter and genes, such as the late embryo specific promoter of DNH1 or the HVA1 promoter and genes; the GLB1 promoter and genes from corn, and any of Zein promoter and genes (Z27) could be used as targets.

Examples of blocking sequences that can be used, include, but are not limited to, non-coding DNA sequences, and/or any plant selectable marker sequence driven by an appropriate promoter sequence in a plant gene expression cassette. Any selectable marker that allows recovery of cells from non-transformed cells in transformation can be used. Particular examples include, but are not limited to: genes that confer resistance to toxic chemicals such as the bar and pat genes which confer herbicide resistance, and those that impart a visually distinguishing characteristic, such as a color change. In addition, any cytotoxic sequence can be used to practice the methods disclosed herein, as long as the gene interferes with floral development, such as pollen or tapetal development, thereby rendering the plant sterile. Particular examples include, but are not limited to ribonucleases, such as barnase, as well as antisense sequences, such as a tapetum-specific antisense gene sequence.

Many workable constitutive or inducible plant promoters can be used. Examples of inducible promoters that can be used to practice the methods disclosed herein include, but are not limited to: heat shock promoters, glucocorticoid promoters, transcriptionally regulated promoters, chemically inducible promoters (MF), and light activated promotes. Promoters regulated by heat shock, such as the promoter associated with the gene encoding the 70-kDa heat shock protein, increase expression several-fold after exposure to elevated temperatures.

In contrast to inducible promoters, constitutive promoters function under most environmental conditions. Many different constitutive promoters can be utilized with respect to the methods of this disclosure. Exemplary constitutive promoters include, but are not limited to, promoters from plant viruses such as the 35S promoter from CaMV; promoters from such plant genes as rice actin; ubiquitin; pEMU; MAS and maize H3 historic and (Atanassova et al. Plant J. 2:291-30.Q0, 1992); and the ALS promoter, a XbaI/NcoI fragment 5′ to the Brassica napus ALS3 structural gene or a nucleotide sequence with substantial sequence similarity. A particular example is a maize ubiquitin gene promoter.

### Example 1

### Expression of Tapetum Specific Disruption in Switchgrass Results in Impaired Male Fertility

This example describes methods used to develop transgenic male impaired fertility switchgrass and provides the basis for targeted gene disruption to cause male sterility. Similar methods can be used to produce other transgenic male sterile perennials. The male sterile plants produced prevent outcrossing thus preventing gene flow in plants such as switchgrass that are obligate outcrossers. Also, male sterility combined with herbicide resistance provides a basic breeding tool allowing the selection of rare outcrossing events between distant heterotic groups. Briefly, switchgrass cells are transformed with DNA sequences that cause herbicide resistance and male sterility using the SL technology. As a control and first proof of concept, a construct a construct comprising the tapetum specific promoter driving the expression of the cytotoxic gene (barnase) has been introduced and analyzed in transgenic switchgrass plant.

### Transformation of Plants

Several systems can be used to transform switchgrass plant cells. The methods disclosed herein are not limited to any particular transformation method. Methods that can be used to transform various grass species (such as switchgrass, creeping bentgrass, tall fescue, perennial ryegrass, Bermuda grass, and Kentucky blue grass) include, but are not limited to, biolistics, Agrobacterium, and whisker-mediated transformation. A strain similar to the Agrobacterium superbinary system was used with a tissue culture approach for selection of bar gene expression in transformed Agrostis pahlstris (cvs Penn A4) and switchgrass (Panicum virgatum L. cv Alamo), cells. The plasmids with gene constructs of interest were introduced into Agrobacterium tumefaciens strains LBA4404 (containing co-integrative vector pSB 111) by triparental mating or electroporation. The two plasmids co-integrate by homologous recombination in Agrobacterium tumefaciens cells.

Mature seeds of creeping bentgrass (cultivars Penn A4) switchgrass (Panicum virgatum L. cv Alamo), were surface-sterilized and plated on callus induction media (modified MMSG or MSA2D media). The plates were kept in the dark at room temperature (RT or 27 C) for 3-6 weeks. The proliferating calli were selected and transferred to new maintenance medium on a regular basis. Only callus that is friable, embryogenic and regenerable is used for transformation. The chosen callus was transferred to fresh medium prior to co-cultivation with Agrobacterium to promote active cell division. This callus was used for transformation within a week after transferring to new plates.

Agrobacterium tumefaciens was induced with acetosyringone as follows: Agrobacterium tumefaciens LBA4404, harboring male sterility vectors were streaked from a glycerol stock and grown at 28° C. on plates containing AB medium, supplemented with 10 gg/ml tetracycline and 50 gg/ml spectinomycin. After three to six days, the cells were scraped from the plate and suspended in Agrobacterium growth medium containing 100 μM acetosyringone, and grown to an OD₆₆₀ of about 0.1-0.5. The bacterial suspension was incubated at 25° C. in the dark with shaking for 3.5 hours before using it for co-cultivation.

Friable callus (0.001 mg-100 g) was mixed with the pre-induced Agrobacterium suspension and incubated at room temperature in the dark for 1.5 hours. The contents were poured into a sterile Buchner-funnel containing a sterile Whatman filter paper. Mild vacuum was applied to drain the excess Agrobacterium suspension. The filter was moved to a plate containing maintenance medium supplemented with 100 μM acetosyringone, and the plate stored in the dark at room temperature for three days. Subsequent to the three day co-cultivation, the co-cultivated calli were rinsed with 250 μg/ml cefotaxime to suppress bacterial growth, and the calli placed on agar plates containing maintenance medium which included 15 mg/L PPT (phosphinothricine, for bar selection) and 250 μg/ml cefotaxime. The calli were kept in the dark at RT for 6-8 weeks and checked periodically for proliferation of the calli on the 15 mg/L PPT. Subsequently, the PPT-resistant calli were placed on regeneration medium containing PPT and cefotaxime. The proliferating calli were first moved to Regeneration Medium I containing cefotaxime (Research Products International Corp.) and PPT (Duchefa Biochemie, B.V.). The tiny plants were separated and transferred to deep peWi plates containing Regeneration Medium II to promote root growth. PPT and cefotaxime were included in the medium to respectively maintain selection pressure and kill any remaining Agrobacterium cells. After 2-3 weeks, or when the plants were 1.5-2 cm tall, they were moved to plant-cons containing MSO II without antibiotics. When the plants were about 10 cm tall and develop extensive root systems, they were transferred to soil and grown for 3-4 weeks with 12 hours light/day. The plants were then transferred to 6-inch pots in the greenhouse, where the temperature is maintained between 21-25° C. Supplemental lighting can be added to increase timing of light exposure for flowering.

### Generation T0 Male Sterile Transformants

The transgenic plants were vegetatively propagated and increased. The TO plants produced seeds by backcrossing to the recipient variety and outcrossing to other cultivars for transmission of the transgenic traits.

Transformants were screened for glufosinate resistance by ‘paint assays’ to leaves and subsequently analyzed by standard molecular procedures (PCR and Southern blotting) to characterize the insertion events in the regenerating TO plants and their stability in subsequent generations. The plants were sprayed with 0-100% v/v of liberty or finale (Aventis Corp.) and shown to be resistant to the herbicide. Southern analysis using the bar gene and/or barnase as probes revealed transgene insertion in the male-sterile plants. Therefore, stable transformation of switchgrass was achieved, as evidenced by the resistance of the plants to herbicide (due to the presence of the bar gene) and the male sterility due to the presence of the male sterile constructs.

### Developmental and Phenotypic Analysis of Pollen Development and Viability

The herbicide-resistant male sterile TO plants had normal vegetative growth and morphology in comparison to non-transgenic tissue culture regenerated plants. As described above, transformation of herbicide tolerant switchgrass (Panicum virgatum L. cv Alamo) was achieved. All transgenic plants were linked to one or the other male sterility constructs (See FIG. 2) as shown by macrophotography and light microscopy. In addition, flowering herbicide resistant male sterile TO plants had normal vegetative growth and morphology in comparison to non-transgenic tissue culture regenerated plants except the anthers were shrunken and the pollen was aborted prior to the starch filling stage as indicted by IKI₂ (iodine) staining. Pollen was obtained from transfected and control plants, and the viability determined by staining with iodine (IKI2) and examination by microscopy, using methods known to those skilled in the art. Wild-type pollen was heavily stained with IKI2, indicating that the pollen was filled with starch and viable. Pollen viability for wild-type plants was between 30-85%. In contrast, transgenic plants (plants transfected with tapetum specific SL constructs, (See FIG. 1) have no visible IKI2 staining, indicating that the pollen was not filled with starch, and thus not viable (See FIG. 3). In addition, fewer pollen grains were observed in the male sterile plants. Pollen fertility was determined using several methods, including in vitro pollen germination analysis, in vivo pollen tube studies, and a fertility test to nontransgenic varieties analyzed for glufosinate resistance.

These plants have been used here to demonstrate the effectiveness of using male sterility and thus provide the basis for creating stable knockout mutations targeting such promoters or genes.

A breeding strategy has been developed (see FIG. 1) to utilize male sterility for the recovery of rare hybrids in switchgrass. Male sterility provides an effective strategy for interrupting gene flow through the pollen. In addition, male sterility may allow for the recovery of rare wide crosses. Promoters from male gametophyte-specific genes, such as Zm13 from maize and rts from rice, can be used to induce male sterility. A gene construct was selected consisting of a rice tapetum-specific promoter, rts, fused to the ribonuclease gene barnase and linked to a constitutive bar cassette for glufosinate resistance. Using Agrobacterium-mediated transformation, this gene construct was successfully introduced into switchgrass (cv Alamo), producing a total of over 96 stably transformed individual events. The vegetative phenotype of the transgenic plants was identical compared with the control wild-type plants indicating that expression of tapetum-specific barnase did not affect normal plant development. T0 plants have been evaluated for herbicide resistance in paint assays; PCR and Southern blots have confirmed transformation. This strategy is useful for recovery of wide crosses and as a gene confinement approach.

Biomass producers are in need of higher-yielding crops that will tolerate climate change and marginal soils not utilized for food crops. Switchgrass is a wind pollinated obligate outcrosser which grows across much of the eastern United States with lowland (warm season) and upland (cool season) varieties. Martinez-Reyna, J. M. and K. P. Vogel (1998, 2008) produced hybrids of and lowland variety cv Kanlow and an upland variety cv Summer which were evaluated for heterosis in field trials over a 3-yr period. Their data indicate that lowland and upland switchgrasses represent different heterotic groups that can potentially be exploited to produce F1 hybrid varieties with improved characteristics (Martinez-Reyna and Vogel, 2008). Controlled hybridizations will become important to the development of new varieties and will be useful for genetic analyses, including those that use molecular markers. A laborious technique using hand emasculation of small grass florets has been previously used to make hybrid switchgrass. The development of improved and regionally selected varieties through conventional breeding will improve yield and contribute to future crop development. To facilitate the new variety development in switchgrass strategy was developed (See FIG. 1) to use herbicide resistant male sterile lines to recover rare wide crosses. Previously a construct (pHG018) has been tested in creeping bentgrass (Luo et al. 2003) which conferred herbicide resistance and events with 100% sterility were observed. This construct contains a rice ubiquitin promoter driving expression of the bar gene conferring resistance to Finale (glufosinate) and a rice tapetum specific promoter driving the expression of barnase (See FIGS. 2A and 2B). In particular, FIG. 2A shows a test construct (PHG 018) for herbicide resistance and nuclear male sterility by tapetal ablation caused by tissue specific expression of barnase. FIG. 2B shows a test construct for SL knockouts. The selectable marker gene does not need to be physically attached and may be on a separate construct. This construct was tested in transgenic switchgrass plants.

TABLE 1
Sample transformation experiment efficiencies cv Alamo

| E | # pieces | # of events | # of plants recovered | % transformation |
|---|---|---|---|---|
| 3 | 400 | 6 | 37 | 1.50% |
| 4 | 600 | 15 | 63 | 2.50% |
| 1 | 577 | 29 | 176 | 5.03% |
| 2 | 588 | 46 | 293 | 7.82% |

Table 1 shows sample efficiencies of transformation experiments inoculating 2165 embryogenic calli with vector PHG 18 and recovery of 96 independent events with regeneration of 569 transgenic plants.

Transgenic T0 switchgrass plants were grown in soil in 10 inch pots in the greenhouse and flowered in January-February 2009. All plants were morphologically normal with respect to leaf, root, shoot and flower development in comparison to wild type non-transgenic plants. Pollen fertility was assayed by IKI staining twice during anthesis of individual florets. Paint assays with 3% Finale confirmed herbicide resistance. DNA samples were taken from mature plants and processed for PCR and Southern blot analysis (See FIGS. 4, 5)

On the basis of the results in Example 1, it was shown that disruption of male floral development can reduce or eliminate pollen development. Therefore, using this example it is realized that disruption of the same or similar genes involved with male floral development by using SL technology swill result in sterile phenotypes.

### Example 2

### Generation of Targeted Mutations Using SL Yields Male Sterile and Female Sterile Plants which Segregate Away from the Selectable Marker Gene

This example describes methods used to develop transgenic male and female plants with impaired fertility and provides the basis for targeted gene disruption to cause hybrid sterility. Similar methods can be used to produce other transgenic male or female sterile perennial plants. In both male and female sterile lines the transgene cassette can be segregated from the disrupted gene target of maintained in the population by selection. The segregated sterile plants can be used for breeding purposes. Thus, sterility combined with herbicide resistance provides a basic breeding tool allowing the selection of rare outcrossing events between distant heterotic groups. The sterile hybrid plants produced from these crosses prevent outcrossing thus preventing gene flow in plants such as switchgrass that are obligate outcrossers as well as seed scatter. Briefly, switchgrass cells are transformed with DNA sequences that cause herbicide resistance and sterility using the SL technology. As a control and first proof of concept, a construct comprising the tapetum specific promoter driving the expression of the cytotoxic gene (barnase) has been introduced and analyzed in transgenic switchgrass plant.

### Transformation of Plants

Several systems can be used to transform switchgrass plant cells. The methods disclosed herein are not limited to any particular transformation method. Methods that can be used to transform various grass species (such as switchgrass, creeping bentgrass, tall fescue, perennial ryegrass, Bermuda grass, and Kentucky blue grass) include, but are not limited to, biolistics, Agrobacterium, and whisker-mediated transformation. A strain similar to the Agrobacterium superbinary system was used with a tissue culture approach for selection of bar gene expression in transformed Agrostis pahlstris (cvs Penn A4) and switchgrass (Panicum virgatum L. cv Alamo), cells. The plasmids with gene constructs of interest were introduced into Agrobacterium tumefaciens strains LBA4404 (containing co-integrative vector pSB 111) by triparental mating or electroporation. The two plasmids co-integrate by homologous recombination in Agrobacterium tumefaciens cells.

Mature seeds of creeping bentgrass (cultivars Penn A4) switchgrass (Panicum virgatum L. cv Alamo), were surface-sterilized and plated on callus induction media (modified MMSG or MSA2D media). The plates were kept in the dark at room temperature (RT or 27 C) for 3-6 weeks. The proliferating calli were selected and transferred to new maintenance medium on a regular basis. Only callus that is friable, embryogenic and regenerable is used for transformation. The chosen callus was transferred to fresh medium prior to co-cultivation with Agrobacterium to promote active cell division. This callus was used for transformation within a week after transferring to new plates.

Agrobacterium tumefaciens was induced with acetosyringone as follows: Agrobacterium tumefaciens LBA4404, harboring male sterility vectors were streaked from a glycerol stock and grown at 28° C. on plates containing AB medium, supplemented with 10 gg/ml tetracycline and 50 gg/ml spectinomycin. After three to six days, the cells were scraped from the plate and suspended in Agrobacterium growth medium containing 100 μM acetosyringone, and grown to an OD₆₆₀ of about 0.1-0.5. The bacterial suspension was incubated at 25° C. in the dark with shaking for 3.5 hours before using it for co-cultivation.

Friable callus (0.001 mg-100 g) was mixed with the pre-induced Agrobacterium suspension and incubated at room temperature in the dark for 1.5 hours. The contents were poured into a sterile Buchner-funnel containing a sterile Whatman filter paper. Mild vacuum was applied to drain the excess Agrobacterium suspension. The filter was moved to a plate containing maintenance medium supplemented with 100 μM acetosyringone, and the plate stored in the dark at room temperature for three days. Subsequent to the three day co-cultivation, the co-cultivated calli were rinsed with 250 μg/ml cefotaxime to suppress bacterial growth, and the calli placed on agar plates containing maintenance medium which included 15 mg/L PPT (phosphinothricine, for bar selection) and 250 μg/ml cefotaxime. The calli were kept in the dark at RT for 6-8 weeks and checked periodically for proliferation of the calli on the 15 mg/L PPT. Subsequently, the PPT-resistant calli were placed on regeneration medium containing PPT and cefotaxime. The proliferating calli were first moved to Regeneration Medium I containing cefotaxime (Research Products International Corp.) and PPT (Duchefa Biochemie, B.V.). The tiny plants were separated and transferred to deep peWi plates containing Regeneration Medium II to promote root growth. PPT and cefotaxime were included in the medium to respectively maintain selection pressure and kill any remaining Agrobacterium cells. After 2-3 weeks, or when the plants were 1.5-2 cm tall, they were moved to plant-cons containing MSO II without antibiotics. When the plants were about 10 cm tall and develop extensive root systems, they were transferred to soil and grown for 3-4 weeks with 12 hours light/day. The plants were then transferred to 6-inch pots in the greenhouse, where the temperature is maintained between 21-25° C. Supplemental lighting can be added to increase timing of light exposure for flowering.

### Developmental and Phenotypic Analysis of Pollen Development and Viability

The herbicide-resistant male and female sterile T0 plants have normal vegetative growth and morphology in comparison to non-transgenic tissue culture regenerated plants. As described above, transformation of herbicide tolerant switchgrass (Panicum virgatum L. cv Alamo) is achieved to deliver these constructs that become randomly inserted into the genome. All transgenic plants are linked to one or the other sterility constructs (FIG. 3). In addition, flowering herbicide resistant sterile T0 plants have normal vegetative growth and morphology in comparison to non-transgenic tissue culture regenerated plants except that in the male, the anthers are shrunken and the pollen is aborted prior to the starch filling stage as indicted by IKI₂ (iodine) staining. Pollen was obtained from transfected and control plants, and the viability determined by staining with iodine (IKI2) and examination by microscopy, using methods known to those skilled in the art. Wild-type pollen was heavily stained with IKI2, indicating that the pollen was filled with starch and viable. Pollen viability for wild-type plants was between 30-85%. In contrast, transgenic plants (plants transfected with tapetum specific SL expression cassette or complex constructs, see FIG. 1) have no visible IKI2 staining, indicating that the pollen was not filled with starch, and thus not viable (See FIG. 3) In addition, fewer pollen grains were observed in the male sterile plants. Pollen fertility was determined using several methods, including in vitro pollen germination analysis, in vivo pollen tube studies, and a fertility test to nontransgenic varieties analyzed for glufosinate resistance. In the female sterile plants, the ovule is aborted but the phenotype of the plant is otherwise normal.

These plants have been used here to demonstrate the effectiveness of using sterility and thus provide the basis for creating stable knockout mutations targeting such promoters or genes. A breeding strategy has been developed to utilize sterility for the recovery of hybrids in switchgrass and other perennial plants. Hybrid sterility provides an effective strategy for interrupting gene flow through the pollen as well as through seed scatter. In addition, hybrid sterility allows for the recovery of inbred populations. Promoters and genes from male gametophyte-specific genes, such as Zm13 from maize and its from rice, can be used to induce male sterility. A gene construct has been selected consisting of a rice tapetum-specific promoter, rts, fused to the ribonuclease gene barnase and linked to a constitutive bar cassette for glufosinate resistance. Using Agrobacterium-mediated transformation, this gene construct has been successfully introduced into switchgrass (cv Alamo), producing a total of over 96 stably transformed individual events. The vegetative phenotype of the transgenic plants was identical compared with the control wild-type plants indicating that expression of tapetum-specific barnase did not affect normal plant development. T0 plants have been evaluated for herbicide resistance in paint assays; PCR and Southern blots have confirmed transformation.

Using this hybrid strategy for the production of sterile hybrids will also allow the mutant to be recovered away from the selectable marker and the SL cassette, resulting in a stable mutation that does not contain a transgene.

### Example 3

In the third example, the method includes contacting a plant, or plant cell, with a vector, wherein the vector includes a construct to express zinc finger nucleases specific to either male or female floral specific gene(s), including those described above, operably linked to a plant promoter and including a gene of interest (GOI) targeted to disrupt the floral specific genes. The plant promoter may be operably linked to a tissue specific promoter or can be constitutively expressed. The production of transgenics may or may not include the use of a selectable marker gene, but the preferred example is using selection. In this example the expression of the SL complex is delayed by the interruption of expression by using the selectable marker as a blocking fragment flanked by site specific recombination sites, such as frt sequences or their mutant derivatives. The advantage of this strategy is that both male and female lines remain fertile until later expressed. This method of producing a sterile perennial plant comprises: crossing a first fertile plant having a desirable trait with second fertile plant, wherein the first fertile plant comprises a first vector comprising a promoter operably linked to a blocking sequence, wherein the blocking sequence is flanked by a recombining site sequence, and a SL complex sequence, wherein the second fertile plant comprises a second vector comprising a promoter operably linked to a recombinase, such as FLP; and thereby permitting expression of the recombinase, wherein crossing the first and second fertile plant results in production of a sterile perennial plant. The same could be done to create male and female sterile lines.

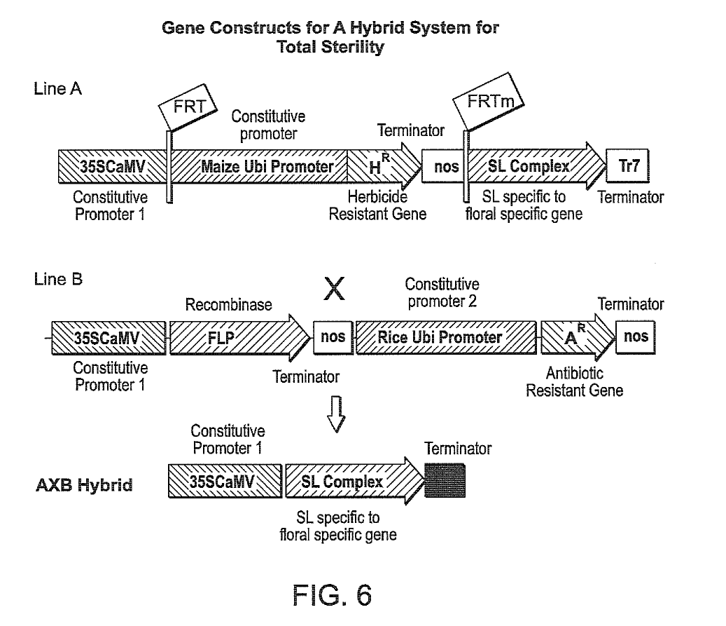

The preferred example would use an herbicide resistance marker in the male, but the female may also be used as well as using two compatible marker to create a doubly selectable hybrid. Expression of this vector results in the production of a seed deficient plant, thereby producing a producing a plant having reduced or no progeny. (See FIG. 6)

Thus generally, the studied method may produce a hybrid perennial plant system for plant breeding of co-sexual plants for increased yields and for having increased gene confinement capabilities includes contacting a hybrid perennial plant with a vector, wherein the vector comprises a SL expression cassette to create a plant line (A) with disrupted male development; (b) contacting a hybrid perennial plant with a vector, wherein the vector comprises a SL expression cassette to create a plant line (B) with disrupted female development; (c) crossing plant line (A) with plant line (B); and (d) producing a perennial plant having increased heterozygocity and gene confinement. The perennial plant may be male sterile plants, female sterile plants or hybrid plants with total gametic sterility. A target sequence of the vector may be male or female specific.

The method produces a perennial plant having a decrease of viable pollen which is less than 0.1% when compared to a wild type perennial plant of a same variety or may even be less than 0.01% when compared to a wild type perennial plant of a same variety. The method also produces a perennial plant with a resulting decrease of the development of viable ovules which produces an amount of viable seed that is less than 0.1% when compared to a wild type perennial plant of a same variety or may even be less than 0.01% when compared to a wild type perennial plant of a same variety.

In light of the foregoing, it will now be appreciated by those skilled in the art that various changes may be made to the embodiment herein chosen for purposes of disclosure without departing from the inventive concept defined by the appended claims. Non limiting examples of such changes including using

```
seq 1
Male female sterility patent Kausch and Dellaporta
LOCUS NM_001148692 2126 bp mRNA linear PLN 10-APR-2009
DEFINITION Zea mays hypothetical protein LOC100274330 (LOC100274330), mRNA.
ACCESSION NM_001148692
VERSION NM_001148692.1 GI: 226497321
KEYWORDS .
SOURCE Zea mays
  ORGANISM Zea mays
        Eukaryota; Viridiplantae; Streptophyta; Embryophyta; Tracheophyta;
        Spermatophyta; Magnoliophyta; Liliopsida; Poales; Poaceae; PACCAD
        clade; Panicoideae; Andropogoneae; Zea.
COMMENT PREDICTED REFSEQ: This record has not been reviewed and the
        function is unknown. The reference sequence was derived from
        BT067785.1.
FEATURES Location/Qualifiers
    source 1 . . . 2126
              /organism = “Zea mays”
              /mol_type = “mRNA”
              /db_xref = “taxon: 4577”
    gene 1 . . . 2126
              /gene = “LOC100274330”
              /note = “hypothetical protein LOC100274330”
              /db_xref = “GeneID: 100274330”
    CDS 17 . . . 1894
              /gene = “LOC100274330”
              /codon_start = 1
              /product = “hypothetical protein LOC100274330”
              /protein_id = “NP_001142164.1”
              /db_xref - “GI: 226497322”
              /db_xref = “GeneID: 100274330”
              /translation = “MTSAGDPISILIPDTQARPRNPRACMLPADAYLRFVFMAAAAYC
CECDVQAAAGTVLQSSGEAIVAGAMGGGVHHHHPCVAADGDGAGAGPGPASVEAALRPLVGVDAWDY
CVYWRLSPDQRFLEMAGFCCSSQFEAQLPALGDLPPSIQLDSSSAGMHAEAMVSNQPIWQSSRVPELQTGY
SSGMVQEPGSSGGPRTRLLVPVAGGLVELFAARYMAEEEQMAELVMAQCGVPSGGEGGAWPPGFAWDG
GASDASRGMYGDAVPPSLSLFDAAGSVAADPFQAVQQAPGAGGGGVDDVAGWQYAAAAGSELEAVQLQ
QEQQPRDADSGSEVSDMQGDPEDDGDGDAQGRGGGKGGGKRQQCKNLEAERKRRKKLNERLYKLRSLV
PNISKMDRAAILGDAIDYIVGLQNQVKALQDELEDPADGAGAPDVLLDHPPPASLVGLENDESPPTSHQHP
LAGTKRARAAAEEEEEEKGNDMEPQVEVRQVEANEFFLQMLCERRPGRFVQIMDSIADLGLEVTNVNVTS
HESLVLNVFRAARRDNEVAVQADRLRDSLLEVMREPYGVWSSSAPPVGMSGSGIADVKHDSVDMKLDGII
DGQAAPSVAVGVSEDHYGGYNHLLQYLA”
ORIGIN
      1 caaatcccac acacccatga catcggccgg cgatccgatc tctatcctga ttcctgacac
    61 acaagcacgg ccacggaacc ctcgcgcatg catgctgcct gctgacgctt accttcgttt
  121 cgttttcatg gctgctgctg cttattgctg cgagtgcgat gtgcaggcgg cagcgggcac
  181 agtcctccag tcctcgggtg aggcgatcgt cgccggcgcc atgggagggg gagtccacca
  241 ccaccacccg tgcgtggctg ctgatggaga tggggccggg gccgggcccg ggccggccag
  301 cgtggaggcc gcgttgaggc ctcttgtcgg cgtcgacgcc tgggactact gcgtctactg
  361 gaggctgtct cctgatcaga ggttcttgga gatggctggg ttctgctgca gcagtcagtt
  421 cgaggcacag cttccagcgc tgggcgacct gcctccatca atccagctcg actcctcgtc
  481 tgcagggatg cacgccgagg caatggtgtc caaccagccg atctggcaga gcagccgcgt
  541 gccagagctc caaacaggtt actccagtgg catggtgcag gagcccgggt ccagcggcgg
  601 cccgaggacg cggctgctgg tgcccgtcgc cggcggcctc gtcgagctct tcgcggcgag
  661 atacatggcg gaggaggagc agatggcgga gctggtgatg gcgcagtgcg gggtgccgag
  721 cggcggtgag gggggcgcgt ggccgccggg attcgcgtgg gacggcggcg cctcggacgc
  781 gtcgcgtggg atgtacggcg atgcggtgcc gccgtcgctc agcctgttcg acgccgccgg
  841 cagcgtcgcg gcggacccgt tccaggcggt gcagcaggcg ccgggcgccg gtggtggtgg
  901 ggtggacgac gtcgccgggt ggcagtatgc tgctgcggct gggagcgagc tggaggcggt
  961 gcagctgcag caggagcagc agccgcgcga tgcggactcg gggtccgagg tcagcgacat
1021 gcagggggac ccagaggacg acggcgacgg cgacgcgcag gggcgtggcg gcggcaaggg
1081 cggcgggaag cggcagcagt gcaagaacct cgaggcggag cggaagcggc ggaagaagct
1141 caacgagcgg ctctacaagc tcaggtcgct cgtcccgaac atctccaaga tggaccgcgc
1201 ggcgatcctc ggggacgcca tcgactacat cgtgggcctg cagaaccagg tgaaggcgct
1261 gcaggacgag ctggaggacc cggcggacgg cgccggcgcc cccgacgtcc tcctggacca
1321 cccgccgccg gcgagcctgg tggggctgga gaacgacgag tcgccgccca cgagccacca
1381 gcacccgctc gccgggacca agagggcccg tgcggcggcg gaggaggagg aggaggagaa
1441 ggggaatgac atggagccgc aggtggaggt gcggcaggtg gaggccaacg agttcttcct
1501 gcagatgctg tgcgagcgcc ggcccgggcg cttcgtccag atcatggact ccatcgccga
1561 cctgggactg gaggtcacca acgtcaacgt cacctcccac gagagcctcg tcctcaacgt
1621 cttccgcgcc gccaggcggg acaatgaggt ggcagtgcag gcggacagac tgagggactc
1681 gctgctggag gtgatgcggg agccgtacgg cgtatggtcg tcgtcggcgc cgcccgtggg
1741 gatgagcggc agcggcatcg ccgacgtgaa gcatgacagc gtggacatga agctcgatgg
1801 catcatcgac gggcaggcgg caccgagcgt cgcagtgggg gtttcagagg atcactacgg
1861 cggctacaac catctcctcc aatacctcgc ttgatcatta tttaattgcg ttcgttcatg
1921 ttgaaagttc gatcaaacta tcaaaggatg gatcaactaa taaaaacggg atccatatat
1981 aagtaactgt gaattgcgat cattaattgt atgcatacaa gcatatggtc gtggattaaa
2041 gtttgttaat tgggttttct cactgctttt ctggatcttt cttgtgttgg ttcaaacgag
2101 ggcggaataa taaagctatt tcctct
//
seq 2
Male female sterility patent Kausch and Dellaporta
LOCUS NM_001148692 2126 bp mRNA linear PLN 10-APR-2009
DEFINITION Zea mays hypothetical protein LOC100274330 (LOC100274330), mRNA.
ACCESSION NM_001148692
VERSION NM_001148692.1 GI: 226497321
KEYWORDS .
SOURCE Zea mays
  ORGANISM Zea mays
        Eukaryota; Viridiplantae; Streptophyta; Embryophyta; Tracheophyta;
        Spermatophyta; Magnoliophyta; Liliopsida; Poales; Poaceae; PACCAD
        clade; Panicoideae; Andropogoneae; Zea.
COMMENT PREDICTED REFSEQ: This record has not been reviewed and the
        function is unknown. The reference sequence was derived from
        BT067785.1.
FEATURES Location/Qualifiers
    source 1 . . . 2126
                    /organism = “Zea mays”
                    /mol_type = “mRNA”
                    /db_xref = “taxon: 4577”
    gene 1 . . . 2126
                    /gene = “LOC100274330”
                    /note = “hypothetical protein LOC100274330”
                    /db_xref = “GeneID: 100274330”
    CDS 17 . . . 1894
                    /gene = “LOC100274330”
                    /codon_start = 1
                    /product = “hypothetical protein LOC100274330”
                    /protein_id = “NP_001142164.1”
                    /db_xref = “GI: 226497322”
                    /db_xref = “GeneID: 100274330”
/translation = “MTSAGDPISILIPDTQARPRNPRACMLPADAYLRFVFMAAAAYCCECDVQAAAGTVLQSSGE
AIVAGAMGGGVHHHHPCVAADGDGAGAGPGPASVEAALRPLVGVDAWDYCVYWRLSPDQRFLEMAGF
CCSSQFEAQLPALGDLPPSIQLDSSSAGMHAEAMVSNQPIWQSSRVPELQTGYSSGMVQEPGSSGGPRTRLL
VPVAGGLVELFAARYMAEEEQMAELVMAQCGVPSGGEGGAWPPGFAWDGGASDASRGMYGDAVPPSLS
LFDAAGSVAADPFQAVQQAPGAGGGGVDDVAGWQYAAAAGSELEAVQLQQEQQPRDADSGSEVSDMQ
GDPEDDGDGDAQGRGGGKGGGKRQQCKNLEAERKRRKKLNERLYKLRSLVPNISKMDRAALLGDAIDYI
VGLQNQVKALQDELEDPADGAGAPDVLLDHPPPASLVGLENDESPPTSHQHPLAGTKRARAAAEEEEEEK
GNDMEPQVEVRQVEANEFFLQMLCERRPGRFVQIMDSIADLGLEVTNVNVTSHESLVLNVFRAARRDNEV
AVQADRLRDSLLEVMREPYGVWSSSAPPVGMSGSGIADVKHDSVDMKLDGIIDGQAAPSVAVGVSEDHY
GGYNHLLQYLA”
ORIGIN
      1 caaatcccac acacccatga catcggccgg cgatccgatc tctatcctga ttcctgacac
    61 acaagcacgg ccacggaacc ctcgcgcatg catgctgcct gctgacgctt accttcgttt
  121 cgttttcatg gctgctgctg cttattgctg cgagtgcgat gtgcaggcgg cagcgggcac
  181 agtcctccag tcctcgggtg aggcgatcgt cgccggcgcc atgggagggg gagtccacca
  241 ccaccacccg tgcgtggctg ctgatggaga tggggccggg gccgggcccg ggccggccag
  301 cgtggaggcc gcgttgaggc ctcttgtcgg cgtcgacgcc tgggactact gcgtctactg
  361 gaggctgtct cctgatcaga ggttcttgga gatggctggg ttctgctgca gcagtcagtt
  421 cgaggcacag cttccagcgc tgggcgacct gcctccatca atccagctcg actcctcgtc
  481 tgcagggatg cacgccgagg caatggtgtc caaccagccg atctggcaga gcagccgcgt
  541 gccagagctc caaacaggtt actccagtgg catggtgcag gagcccgggt ccagcggcgg
  601 cccgaggacg cggctgctgg tgcccgtcgc cggcggcctc gtcgagctct tcgcggcgag
  661 atacatggcg gaggaggagc agatggcgga gctggtgatg gcgcagtgcg gggtgccgag
  721 cggcggtgag gggggcgcgt ggccgccggg attcgcgtgg gacggcggcg cctcggacgc
  781 gtcgcgtggg atgtacggcg atgcggtgcc gccgtcgctc agcctgttcg acgccgccgg
  841 cagcgtcgcg gcggacccgt tccaggcggt gcagcaggcg ccgggcgccg gtggtggtgg
  901 ggtggacgac gtcgccgggt ggcagtatgc tgctgcggct gggagcgagc tggaggcggt
  961 gcagctgcag caggagcagc agccgcgcga tgcggactcg gggtccgagg tcagcgacat
1021 gcagggggac ccagaggacg acggcgacgg cgacgcgcag gggcgtggcg gcggcaaggg
1081 cggcgggaag cggcagcagt gcaagaacct cgaggcggag cggaagcggc ggaagaagct
1141 caacgagcgg ctctacaagc tcaggtcgct cgtcccgaac atctccaaga tggaccgcgc
1201 ggcgatcctc ggggacgcca tcgactacat cgtgggcctg cagaaccagg tgaaggcgct
1261 gcaggacgag ctggaggacc cggcggacgg cgccggcgcc cccgacgtcc tcctggacca
1321 cccgccgccg gcgagcctgg tggggctgga gaacgacgag tcgccgccca cgagccacca
1381 gcacccgctc gccgggacca agagggcccg tgcggcggcg gaggaggagg aggaggagaa
1441 ggggaatgac atggagccgc aggtggaggt gcggcaggtg gaggccaacg agttcttcct
1501 gcagatgctg tgcgagcgcc ggcccgggcg cttcgtccag atcatggact ccatcgccga
1561 cctgggactg gaggtcacca acgtcaacgt cacctcccac gagagcctcg tcctcaacgt
1621 cttccgcgcc gccaggcggg acaatgaggt ggcagtgcag gcggacagac tgagggactc
1681 gctgctggag gtgatgcggg agccgtacgg cgtatggtcg tcgtcggcgc cgcccgtggg
1741 gatgagcggc agcggcatcg ccgacgtgaa gcatgacagc gtggacatga agctcgatgg
1801 catcatcgac gggcaggcgg caccgagcgt cgcagtgggg gtttcagagg atcactacgg
1861 cggctacaac catctcctcc aatacctcgc ttgatcatta tttaattgcg ttcgttcatg
1921 ttgaaagttc gatcaaacta tcaaaggatg gatcaactaa taaaaacggg atccatatat
1981 aagtaactgt gaattgcgat cattaattgt atgcatacaa gcatatggtc gtggattaaa
2041 gtttgttaat tgggttttct cactgctttt ctggatcttt cttgtgttgg ttcaaacgag
2101 ggcggaataa taaagctatt tcctct
//
seq 3
Male female sterility patent Kausch and Dellaporta
LOCUS NM_001148692 2126 bp mRNA linear PLN 10-APR-2009
DEFINITION Zea mays hypothetical protein LOC100274330 (LOC100274330), mRNA.
ACCESSION NM_001148692
VERSION NM_001148692.1 GI: 226497321
KEYWORDS .
SOURCE Zea mays
  ORGANISM Zea mays
        Eukaryota; Viridiplantae; Streptophyta; Embryophyta; Tracheophyta;
        Spennatophyta; Magnoliophyta; Liliopsida; Poales; Poaceae; PACCAD
        clade; Panicoideae; Andropogoneae; Zea.
COMMENT PREDICTED REFSEQ: This record has not been reviewed and the
        function is unknown. The reference sequence was derived from
        BT067785.1.
FEATURES Location/Qualifiers
    source 1 . . . 2126
                  /organism = “Zea mays”
                  /mol_type = “mRNA”
                  /db_xref = “taxon: 4577”
    gene 1 . . . 2126
                  /gene = “LOC100274330”
                  /note = “hypothetical protein LOC100274330”
                  /db_xref = “GeneID: 100274330”
    CDS 17 . . . 1894
                  /gene = “LOC100274330”
                  /codon_start = 1
                  /product = “hypothetical protein LOC100274330”
                  /protein_id = “NP_001142164.1”
                  /db_xref = “GI: 22497322”
                  /db_xref = “GeneID: 100274330”
                  /translation = “MTSAGDPISILIPDTQARPRNPRACMLPADAYLRFVFMAAAAYC
CECDVQAAAGTVLQSSGEAIVAGAMGGGVHHHHPCVAADGDGAGAGPGPASVEAALRPLVGVDAWDY
CVYWRLSPDQRFLEMAGFCCSSQFEAQLPALGDLPPSIQLDSSSAGMHAEAMVSNQPIWQSSRVPELQTGY
SSGMVQEPGSSGGPRTRLLVPVAGGLVELFAARYMAEEEQMAELVMAQCGVPSGGEGGAWPPGFAWDG
GASDASRGMYGDAVPPSLSLFDAAGSVAADPFQAVQQAPGAGGGGVDDVAGWQYAAAAGSELEAVQLQ
QEQQPRDADSGSEVSDMQGDPEDDGDGDAQGRGGGKGGGKRQQCKNLEAERKRRKKLNERLYKLRSLV
PNISKMDRAAILGDAIDYIVGLQNQVKALQDELEDPADGAGAPDVLLDHPPPASLVGLENDESPPTSHQHP
LAGTKRARAAAEEEEEEKGNDMEPQVEVRQVEANEFFLQMLCERRPGRFVQIMDSIADLGLEVTNVNVTS
HESLVLNVFRAARRDNEVAVQADRLRDSLLEVMREPYGVWSSSAPPVGMSGSGIADVKHDSVDMKLDGII
DGQAAPSVAVGVSEDHYGGYNHLLQYL A”
ORIGIN
      1 caaatcccac acacccatga catcggccgg cgatccgatc tctatcctga ttcctgacac
    61 acaagcacgg ccacggaacc ctcgcgcatg catgctgcct gctgacgctt accttcgttt
  121 cgttttcatg gctgctgctg cttattgctg cgagtgcgat gtgcaggcgg cagcgggcac
  181 agtcctccag tcctcgggtg aggcgatcgt cgccggcgcc atgggagggg gagtccacca
  241 ccaccacccg tgcgtggctg ctgatggaga tggggccggg gccgggcccg ggccggccag
  301 cgtggaggcc gcgttgaggc ctcttgtcgg cgtcgacgcc tgggactact gcgtctactg
  361 gaggctgtct cctgatcaga ggttcttgga gatggctggg ttctgctgca gcagtcagtt
  421 cgaggcacag cttccagcgc tgggcgacct gcctccatca atccagctcg actcctcgtc
  481 tgcagggatg cacgccgagg caatggtgtc caaccagccg atctggcaga gcagccgcgt
  541 gccagagctc caaacaggtt actccagtgg catggtgcag gagcccgggt ccagcggcgg
  601 cccgaggacg cggctgctgg tgcccgtcgc cggcggcctc gtcgagctct tcgcggcgag
  661 atacatggcg gaggaggagc agatggcgga gctggtgatg gcgcagtgcg gggtgccgag
  721 cggcggtgag gggggcgcgt ggccgccggg attcgcgtgg gacggcggcg cctcggacgc
  781 gtcgcgtggg atgtacggcg atgcggtgcc gccgtcgctc agcctgttcg acgccgccgg
  841 cagcgtcgcg gcggacccgt tccaggcggt gcagcaggcg ccgggcgccg gtggtggtgg
  901 ggtggacgac gtcgccgggt ggcagtatgc tgctgcggct gggagcgagc tggaggcggt
  961 gcagctgcag caggagcagc agccgcgcga tgcggactcg gggtccgagg tcagcgacat
1021 gcagggggac ccagaggacg acggcgacgg cgacgcgcag gggcgtggcg gcggcaaggg
1081 cggcgggaag cggcagcagt gcaagaacct cgaggcggag cggaagcggc ggaagaagct
1141 caacgagcgg ctctacaagc tcaggtcgct cgtcccgaac atctccaaga tggaccgcgc
1201 ggcgatcctc ggggacgcca tcgactacat cgtgggcctg cagaaccagg tgaaggcgct
1261 gcaggacgag ctggaggacc cggcggacgg cgccggcgcc cccgacgtcc tcctggacca
1321 cccgccgccg gcgagcctgg tggggctgga gaacgacgag tcgccgccca cgagccacca
1381 gcacccgctc gccgggacca agagggcccg tgcggcggcg gaggaggagg aggaggagaa
1441 ggggaatgac atggagccgc aggtggaggt gcggcaggtg gaggccaacg agttcttcct
1501 gcagatgctg tgcgagcgcc ggcccgggcg cttcgtccag atcatggact ccatcgccga
1561 cctgggactg gaggtcacca acgtcaacgt cacctcccac gagagcctcg tcctcaacgt
1621 cttccgcgcc gccaggcggg acaatgaggt ggcagtgcag gcggacagac tgagggactc
1681 gctgctggag gtgatgcggg agccgtacgg cgtatggtcg tcgtcggcgc cgcccgtggg
1741 gatgagcggc agcggcatcg ccgacgtgaa gcatgacagc gtggacatga agctcgatgg
1801 catcatcgac gggcaggcgg caccgagcgt cgcagtgggg gtttcagagg atcactacgg
1861 cggctacaac catctcctcc aatacctcgc ttgatcatta tttaattgcg ttcgttcatg
1921 ttgaaagttc gatcaaacta tcaaaggatg gatcaactaa taaaaacggg atccatatat
1981 aagtaactgt gaattgcgat cattaattgt atgcatacaa gcatatggtc gtggattaaa
2041 gtttgttaat tgggttttct cactgctttt ctggatcttt cttgtgttgg ttcaaacgag
2101 ggcggaataa taaagctatt tcctct
//
seq 4
Male female sterility patent Kausch and Dellaporta
LOCUS NM_001165758 2083 bp mRNA linear PLN 25-SEP-2009
DEFINITION Zea mays hypothetical protein LOC100304316 (LOC100304316), mRNA.
ACCESSION NM_001165758
VERSION NM_601165758.1 GI: 259490652
KEYWORDS .
SOURCE Zea mays
  ORGANISM Zea mays
        Eukaryota; Viridiplantae; Streptophyta; Embryophyta; Tracheophyta;
        Spermatophyta; Magnoliophyta; Liliopsida; Poales; Poaceae; PACCAD
        clade; Panicoideae; Andropogoneae; Zea.
COMMENT PREDICTED REFSEQ: This record has not been reviewed and the
        function is unknown. The reference sequence was derived from
        BT060822.1.
FEATURES Location/Qualifiers
    source 1 . . . 2083
                  /organism = “Zea mays”
                  /mol_type = “mRNA”
                  /db_xref = “taxon: 4577”
    gene 1 . . . 2083
                  /gene = “LOC100304316”
                  /note = “hypothetical protein LOC100304316”
                  /db_xref = “GeneID: 100304316”
    CDS 278 . . . 2053
                  /gene = “LOC100304316”
                  /codon_start = 1
                  /product = “hypothetical protein LOC100304316”
                  /protein_id = “NP_001159230.1”
                  /db_xref = “GI: 259490653”
                  /db_xref = “GeneID: 100304316”
                  /translation = “MPIGWSRHPVCGRRYHFIIRNEYKTCKHCDHCGLMAQSFGTRCP
TCKYVISSDDPEDWDYRQLDNPRHLLHGIVHDNGFGHLVRINGREGGSSLLTGIQLMGFWDWLCRYLRVR
KVSLMDVSKKYETDYRILHAITTGHSWYGQWGFKLNKGSFGITSEEYLKAMDNLSLTPLSHFFPHSRYPRN
QLQDTISFYRSLSKQPLTTIRELFLYVLGLATSKSSNMHYGSMEKEHSHTHVQDTWPDEEIKRATEIAIKVL
RAVEKTRWVTMRILKAAMYHSIGSPQLVDYCLKTLGTRTIDGMMVAVRCNSDTNTLEYRLMDEPIVLPNV
SMPTQDHLRRDIKFLHDALLHPHTMHPYKPENCYEHGKRSAMVLLDCKQFTKHYDLEQEFLPQNPSMLHL
WCQVEVLDQVGDPPCIPPELLTLPQTATVSDLKVEATRTFRGIYLMLHSFVADRLVDCGTASESTQLKLLF
GANGTVRVQGRCASGERRVGIYRMERGVDKWTVRCSCGAKDDDGERMLSCDSCHVWQHTRCVGISDFD
QVPKKYVCNSCKLLNKRKSRGHGPVYNIGPSKRFKIGAGGFSSRWGIFLRPPADM”
ORIGIN
      1 ggctgccgcc gcgtggtgcg tttcgcgact gtgtacggtc gttcctggct ggatccgcgg
    61 tgccggcgga cggcgcgtgg caggtagcct ttggagtcgg gaacggggtg gtcgtggtga
  121 tggaggtggt ggaagaggac gtcgctaaaa ctgggatcga gcggatctac tgcgatcact
  181 gtaccgtcgc cgacttgaca gcctgactcc tcattaacct gctcggcctg cttctgtgtg
  241 ttcgtctgaa catgtgtata cattttttct tctacagatg ccaatcggct ggagtagaca
  301 ccctgtttgt ggaaggaggt accatttcat aattcggaat gaatacaaaa cctgcaagca
  361 ttgtgaccat tgtggtctta tggcccagtc gtttggaaca cggtgcccga catgcaaata
  421 tgtgatctcc tctgatgatc cggaagattg ggactatagg cagttggata atccacgtca
  481 cttgctgcat ggtattgtac atgacaatgg gtttggtcac cttgttcgga taaatggcag
  541 agagggtggc tctagtcttc tgacggggat tcaactgatg ggtttctggg attggctctg
  601 cagatacctt agagtcagaa aggtctcctt gatggatgtc tctaagaagt atgaaacaga
  661 ttaccggatc ttacatgcca tcactactgg tcattcatgg tatggccaat ggggattcaa
  721 actcaacaaa gggagctttg gaattacatc agaagaatac ttaaaagcta tggacaacct
  781 ttccttaact ccattatcac acttcttccc gcactcccga tatcctcgaa accagctaca
  841 agataccatt tcattctacc gatctctttc aaagcaacct ctcaccacaa ttcgtgaact
  901 gttcctctat gtgctgggcc ttgccaccag caagagttca aatatgcact atggatcaat
  961 gcataaggag cactcacata cccatgtgca agacacatgg cctgacgagg aaataaaacg
1021 tgcaacagaa attgctataa aggttcttcg tgctgttgag aaaacaaggt gggtgaccat
1081 gcgaatccta aaggcagcca tgtaccattc aattggttca ccgcagctag tggactactg
1141 cctcaagacc cttggtacta gaacaattga tggaatgatg gttgcagttc gatgcaacag
1201 cgatactaac accctagagt acaggcttat ggatgaaccc atcgttctgc ccaatgtatc
1261 catgccaact caagaccatc ttcgccgtga cataaagttc ttgcatgatg ctctcctcca
1321 cccacataca atgcatccat acaaaccgga aaactgttat gagcatggca agaggtctgc
1381 catggtcctt ttggactgca agcaattcac aaagcactat gacctggaac aggagttctt
1441 gcctcaaaac ccatccatgt tgcacctgtg gtgtcaagta gaggtgttag accaggttgg
1501 cgatccacct tgcataccac cagagctcct aactcttccg cagacagcaa ctgtgtctga
1561 tctgaaggtg gaggcaacca gaacattccg tggcatctat ctaatgttgc attcctttgt
1621 agccgatcgg cttgttgact gtggaacggc aagtgagtca actcaactaa agctcttgtt
1681 tggggcaaat ggaactgttc gcgtccaagg caggtgtgcc agtggtgaac gcagggttgg
1741 gatttaccgg atggagagag gcgtggataa atggacagtg cgttgctctt gtggagccaa
1801 ggatgatgat ggtgagagga tgctgtcttg tgactcttgc catgtgtggc agcacactag
1861 gtgtgttggg attagtgatt tcgatcaggt gcccaagaaa tatgtatgta actcatgtaa
1921 attacttaac aagcgtaaga gcagaggtca cggaccagtt tataacattg gcccaagcaa
1981 aagattcaag attggcgcag gtggctttag ctctaggtgg gggatttttt tgaggcctcc
2041 agctgacatg taaatatcaa taaaatgaca gtgagtttgt atg
//
seq 5
Male female sterility patent Kausch and Dellaporta
LOCUS NM_001156998 1410 bp mRNA linear PLN 10-APR-2009
DEFINITION Zea mays DNA binding protein (LOC100284100), mRNA.
ACCESSION NM_001156998
VERSION NM_001156998.1 GI: 226503250
KEYWORDS .
SOURCE Zea mays
  ORGANISM Zea mays
        Eukaryota; Viridiplantae; Streptophyta; Embryophyta; Tracheophyta;
        Spermatophyta; Magnoliophyta; Liliopsida; Poales; Poaceae; PACCAD
        clade; Panicoideae; Andropogoneae; Zea.
REFERENCE 1 (bases 1 to 1410)
  AUTHORS Alexandrov, N. N., Brover, V. V., Freidin, S., Troukhan, M. E.,
          Tatarinova, T. V., Zhang, H., Swaller, T. J., Lu, Y. P., Bouck, J.,
          Flavell, R. B. and Feldmann, K. A.
  TITLE Insights into corn genes derived from large-scale cDNA sequencing
  JOURNAL Plant Mol. Biol. 69 (1-2), 179-194 (2009)
  PUBMED 18937034
COMMENT PROVISIONAL REFSEQ: This record has not yet been subject to final
        NCBI review. The reference sequence was derived from EU967089.1.
FEATURES Location/Qualifiers
    source 1 . . . 1410
                  /organism = “Zea mays”
                  /mol_type = “mRNA”
                  /db_xref = “taxon: 4577”
    gene 1 . . . 1410
                  /gene = “LOC100284100”
                  /note = “DNA binding protein”
                  /db_xref = “GeneID: 100284100”
    CDS 255 . . . 1214
                  /gene = “LOC100284100”
                  /codon_start = 1
                  /product = “DNA binding protein”
                  /protein_id = “NP 001150470.1”
                  /db_xref = “GI: 22503251”
                  /dbxref = “GeneID: 100284100”
/translation = “MGRPPCCDKANVKKGPWTPEEDAKLLAYTSTHGTGNWTNVPQRAGLKRCGKSCRLRYTNY
LRPNLKHENFTQEEEDLIVTLHAMLGSRWSLIANQLPGRTDNDVKNYWNTKLSKKLRQRGIDPLTHRPIAD
LMHSIGALAIRPPQPATSPNGSAAYLPAPALPLVHDVAYHAAGMLPPTPAPPRQVVIARVEADAPASPTEHG
HELKWSDFLADDAAAAAAAAAEAQQQLAVVGQYHHEANAGSSSAAAGGNDGCGIAVGGDDGAAAFID
AILDCDKETGVDQLIAELLADPAYYAGSSSSSSSSSGMGWAGMGLLNAD”
ORIGIN
      1 actaaccatg ccgtggctag ttaaatgacg gggacggggt cacgccttcg ttgcgtgcct
    61 ccacctcccc ccctcggcgc ccccaacgac atgttgttac cgtggctgtg gcagccggcc
  121 ggtctccttc tccatccata tgtactggca gcatcgtatc accttttttt ctgcagcggt
  181 gatctcatct aggcgtcggt cagagctctc tcgagctcgc cagcggtggt tggtcgtcgt
  241 cgtcgtcgtc gtcgatgggg aggccgccgt gctgcgacaa ggcgaacgtg aagaaggggc
  301 cgtggacgcc ggaggaggac gccaagctgc tggcctacac ctccacccat ggcaccggca
  361 actggaccaa cgtgccccaa cgagcagggc tcaagaggtg cggcaagagc tgcaggctga
  421 ggtacaccaa ctacctgcgt cccaacctga agcacgagaa cttcacccag gaggaggaag
  481 acctcatcgt caccctccac gccatgctcg gaagcaggtg gtctctgatc gcgaaccagc
  541 tgccgggaag gacggacaac gacgtgaaga actactggaa cacgaagctg agcaagaagc
  601 tgcggcagcg cgggatcgac cccctcaccc accgccccat cgccgacctc atgcacagca
  661 tcggcgcgct ggccatccgc ccgccgcagc cggcgacctc ccctaacggc tccgccgcct
  721 accttcctgc gccggcgctc ccgctcgtcc acgacgtcgc gtaccacgcc gccggaatgc
  781 tgccgccgac gccggcgccg ccccggcagg tcgtcatcgc gcgcgtggaa gcggacgcgc
  841 ccgcgtcgcc gacggagcac gggcacgagc tcaagtggag cgacttcctc gccgacgacg
  901 ccgccgccgc ggcggcggcc gcggccgagg cgcagcagca gctggccgtt gttgggcagt
  961 accaccacga ggccaacgcc gggagcagca gcgctgcggc cggcggtaac gacggttgtg
1021 gcattgccgt cggcggcgac gacggcgcag cggcgttcat cgacgccatc ctggactgcg
1081 acaaggagac gggggtggac cagctcatcg ccgagctgct ggccgacccg gcctactacg
1141 cgggctcctc ctcctcctcc tcctcctcgt ccgggatggg ctgggccggc atgggcctgc
1201 tgaacgctga ttaattaact caagactgct ttagtgtttg ctatacgtac ttaccatcaa
1261 ttagtatgat ggtcaaacct tccaaccgga tccattcata tgcttgcaca actctgggag
1321 tctgggtgtt ttcggattac aaattgtacg gataattgac gccatttgtg cgtgtgtgtc
1381 tcattcattt tccgaaaaaa aaaaaaaaaa
//
seq 6
Male female sterility patent Kausch and Dellaporta
LOCUS AY733074 2004 bp DNA linear PLN 29-JAN-2005
DEFINITION Zea mays egg apparatus 1 gene, complete cds.
ACCESSION AY733074
VERSION AY733074.1 GI: 57903632
KEYWORDS .
SOURCE Zea mays
  ORGANISM Zea mays
        Eukaryota; Viridiplantae; Streptophyta; Embryophyta; Tracheophyta;
        Spermatophyta; Magnoliophyta; Liliopsida; Poales; Poaceae; PACCAD
        clade; Panicoideae; Andropogoneae; Zea.
REFERENCE 1 (bases I to 2004)
  AUTHORS Marton, M. L., Cordts, S., Broadhvest, J. and Dresselhaus, T.
  TITLE Micropylar pollen tube guidance by egg apparatus 1 of maize
  JOURNAL Science 307 (5709), 573-576 (2005)
  PUBMED 15681383
REFERENCE 2 (bases 1 to 2004)
  AUTHORS Marton, M. L. and Dresselhaus, T.
  TITLE Direct Submission
  JOURNAL Submitted (25-AUG-2004) Developmental Biology & Biotechnology,
          Biocenter Klein Flottbek, University of Hamburg, Ohnhorststrasse
          18, Hamburg 22609, Germany
FEATURES Location/Qualifiers
    source 1 . . . 2004
                  /organism = “Zea mays”
                  /mol_type = “genomic DNA”
                  /db_xref = “taxon: 4577”
                  /chromosome = “7”
                  /map = “7q119-q125”
                  /cell_type = “egg cell”
                  /note = “genotype: A188”
    TATA_signal 1433 . . . 1439
    mRNA 1464 . . . 2004
                  /product = “egg apparatus 1”
    5′UTR 1464 . . . 1570
    CDS 1571 . . . 1855
                  /note = “EA1; functions in micropylar pollen tube guidance”
                  /codon_start = 1
                  /product = “egg apparatus 1”
                  /protein_id = “AAW58117.1”
                  /db_xref = “GI: 57903633”
/translation = “MSSCPAIVNMKDDDGIGAMGAAVAFAAMGVFGIYFLWPVVGPTSAGMMMKAPGAAGWVI
CRAVFEANPQLYFTILRTAGAAAAAATFAACSIAS”
    3′UTR 1856 . . . 2004
ORIGIN
      1 tccacacgat tctgcctgca tattcgtcca aacgactcaa gtcaaatgaa aagaacaatt
    61 ttataactaa aattcgagtc aaatgcattt aatcttgagg ggttacctaa ccctggtgcg
  121 cgagggatgt cgattgtgcg gattgacatg gtaaggtact cttggtcctc atcagcgccc
  181 ttcttcctgt tcgtcggttc gtccgaggtt cgtccttgtg ggtgcgtgtc caagtgaact
  241 caacttcgtc caacccttct ctgtgttttt tcgtccgtca tccttgcctg gggacaaccc
  301 ctccctttta taggtcggga gaggggtcgc ccagcgatgg cttccttagg aaggagttgt
  361 aaggcaaagg taaaaccaac gttctacagg ggtaaagcca cgcgtactcg tgggcccgta
  421 gttgcctaga tgtctcgtat tcacggtggc gaacggcgtg gggctacagg gcccccaacc
  481 gccatcattt aggctatgcc gacccatggc cttcgcagcc tagggctcaa ggcggctcgt
  541 cgcgttgcgc cctgccagtg ttgtgcgcac tcaggtcgag gggacgcaga ctataaatgt
  601 gtcacagtcc gggaggctcg caggtcatga gtgctatgcg atccaagagt tttcgtatgt
  661 catgagtgga aaacggacca atgctcgcgt tgtggctcag actattcatg cggtcggtta
  721 tttatggcgg cttgatctag ggtcacgcgt gggatccact caggtggttt tccttcgaca
  781 tgctcggccc tcctatcagg tttcgacgtc cgaccctggt ctcggtaacg tggtgtttga
  841 ccggggacaa gctcttttag agttgacgca tccatctctt ccagctgacc aacggatcta
  901 gcgactaggg ctcttcgtta gcgtggtcag agacgtgttc tcttaccggc ggatcatttt
  961 ccgacaatac taatccaaag gcaggctcat ggtggtacat gtccaagtcc aatcttctaa
1021 tgggtatagt taggttattt aaaataatac cctaaattct gtcactttct tcattttaat
1081 actaatccaa gctgccacga cggattgctg gtagtggacg agtagtatcg gcaaaaaata
1141 attactactt tttttccggt aaaatttgat tactactcta cataattagc aaatgaaatt
1201 aatcacctct tatgcacgtt ctcactagta ccaagcaaca attcagcttc tgcatttcgc
1261 taccgttctc ttcaatgcgc tcgactgatc gcgcacattg cgaagctgtc tcttcgtcgt
1321 ggcctgccat tgggattcga gacggggagc aaatgcgcac ggcatgcatc gcaatgcagg
1381 caatgaagcc gagcagacgc ctggccaacc tcgatacggc gctgcagcct actacaaata
1441 gatgcccaat taacacaaca cgcagcgccc gctgtccatt cattcaaaac ccagccgatc
1501 gctctcctcc aactaagcag caagggcaga agcaacgccg gcgtgcccca cggacgacgc
1561 tgaattctgc atgtcatcct gcccggccat cgtcaacatg aaggacgacg atggcatagg
1621 cgctatggga gcggcggtgg cgtttgccgc catgggcgtc ttcggcatct atttcctgtg
1681 gcccgtggtg ggccccactt cggcggggat gatgatgaag gcgcccggcg ccgcagggtg
1741 ggtcatctgc cgcgcggtgt tcgaggccaa cccgcagttg tattttacca tcctccgcac
1801 ggccggcgcg gcagctgccg ctgccacgtt cgctgcctgt tcgatcgcta gctagcgcta
1861 gctgtgactg tgagcaagtg atcgtcgtaa ataaaagata gcgagcgacg agacgagcag
1921 catctgccag tatttccgcc gtatgccgat gttgtcggtg ttttcccatt gaatggagat
1981 gttactctat gcgtcgtaat tgcc
//
```

We claim:

1. A method of producing a hybrid perennial plant system for plant breeding of co-sexual plants for increased yields and for having increased gene confinement capabilities comprising:
(a) contacting a first compatible perennial plant with a male vector, wherein the male vector comprises a SL expression cassette to create a plant line (A) with disrupted male development;
(b) contacting a second compatible perennial plant with a female vector, wherein the female vector comprises a SL expression cassette to create a plant line (B) with disrupted female development; and
(c) crossing plant line (A) with plant line (B) to produce the hybrid perennial plant having increased heterozygocity gene confinement.

2. The method of claim 1, wherein the first compatible perennial plant with the male vector is a member selected from the group consisting of male sterile plants, female sterile plants and hybrid plants with total gametic sterility.

3. The method of claim 1, wherein a target sequence of the male vector is male specific.

4. The method of claim 1, wherein a target sequence of the female vector is female specific.

5. The method of claim 2, wherein the SL expression cassette is operably linked to an herbicide selectable marker.

6. The method of claim 5, wherein the herbicide selectable marker is selected such that contacting the hybrid plant with the vector creates the perennial plant line (A) which is male sterile.

7. The method of claim 5, wherein the herbicide selectable marker is selected such that contacting the hybrid plant with the vector creates the perennial plant line (B) which is female sterile and is other than the herbicide selectable marker selected such that contacting the hybrid plant with the vector creates the perennial plant line (A) which is male sterile.

8. The method of claim 1, wherein the perennial plant line (A) and the perennial plant line (B) each contain a transgene cassette that is able to be segregated from a disrupted gene target.

9. The method of claim 1, wherein the perennial plant line (A) and the perennial plant line (B) each contain a transgene cassette that is maintained in a population by selection.

10. The method of claim 2, wherein the hybrid plants contain both disrupted male and female reproductive sequences.

11. The method of claim 1, wherein the SL expression cassette is operably linked to an herbicide selectable marker.

12. The method of claim 1, wherein the SL expression cassette is operably linked to an herbicide selectable marker to create and maintain line (A) as male sterile.

13. The method of claim 1, wherein the SL expression cassette is operably linked to an herbicide selectable marker different from that isn line (A) to create and maintain line (B) as female sterile.

14. The method of claim 1, wherein the method produces a perennial plant with a resulting in a decrease of viable pollen.

15. The method of claim 14, wherein the resulting decrease in viable pollen is less than 0.1% when compared to a wild type perennial plant of a same variety.

16. The method of claim 14, wherein the resulting decrease in viable pollen is less than 0.01% when compared to a wild type perennial plant of a same variety.

17. The method of claim 1, wherein the method produces a perennial plant with a resulting decrease of the development of viable ovules.

18. The method of claim 17, wherein the resulting decrease of the development of viable ovules produces an amount of viable seed that is less than 0.1% when compared to a wild type perennial plant of a same variety.

19. The method of claim 17, wherein the resulting decrease of the development of viable ovules produces an amount of viable seed that is less than 0.01% when compared to a wild type perennial plant of a same variety.

20. The method of claim 1, wherein the perennial plant contains a desirable trait selected from the group consisting of herbicide resistance, drought tolerance and disease tolerance.

21. The method of claim 20, wherein the desired trait is operably linked to the perennial plant having increased gene confinement.

22. The method of claim 1 wherein the increased gene confinement is further propagated through vegetative reproduction.

23. The method of claim 1 wherein the SL expression cassette is ZFN.

24. A perennial plant produced by the method of claim 1.

25. A male sterile perennial plant produced by the method of claim 1.

26. A seed produced from a perennial plant produced by the method of claim 2.

27. The method of claim 1, wherein contacting the plant comprises transforming a gene of the plant with the vector.

28. A method of producing a sterile hybrid perennial plant, comprising:
(a) obtaining a male sterile plant having desirable traits selected from the group from the group consisting of herbicide resistance, drought tolerance and disease tolerance;
(b) obtaining a female sterile plant;
(c) crossing the male sterile plant with the female sterile plant; and
(d) producing the sterile hybrid perennial plant wherein, the male sterile plant contains a first vector comprising a male sterile knockout mutation created using a SL expression cassette and the female sterile plant contains a second vector comprising a female sterile knockout mutation created using a SL expression cassette wherein crossing the first and second plants results in production of a sterile perennial plant.

29. The method of claim 28 wherein the SL expression cassette is ZFN.
